(19) United States
(12) Patent Application Publication
(10) Pub. No.: US 2017/0065346 A1
(43) Pub. Date: Mar. 9, 2017

(54) TREATMENT DEVICES AND REALTIME INDICATIONS

(71) Applicant: ASYMMETRIC MEDICAL LTD., Kfar Mordechai (IL)

(72) Inventors: Ori Weisberg, Kfar Mordechai (IL); Moshe Eshkol, Harutzim (IL); Summer-Galai Amit, Tel Aviv (IL)

(21) Appl. No.: 15/119,415

(22) PCT Filed: Feb. 16, 2015
(86) PCT No.: PCT/IL2015/050173

§ 371 Date: Aug. 17, 2016

Related U.S. Application Data
(60) Provisional application No. 61/940,518, filed on Feb. 17, 2014.

(51) Int. Cl. A61B 18/22; F21V 8/00; A61B 90/00; A61B 90/30; A61B 42/10; A61B 42/20

(52) U.S. Cl. CPC A61B 18/22; A61B 42/10; A61B 42/20; A61B 90/08; A61B 90/30; A61B 90/39; G02B 6/001; A61B 2017/00026

(57) ABSTRACT

Devices and methods are provided for indicating treatment efficiency by illuminating a signal onto surrounding tissue. The signal may be dynamic and is configured to indicate specific parameters of the treatment, the device and the patient. Various signal features may denote different parameters. Devices and methods are provided for maintaining a fiber tip effective throughout a treatment procedure by regenerating or replacing the tip. Devices and methods are provided for controlling emission from bended fibers. Cordless configurations of laser treatment devices are also provided.

### BACKGROUND OF THE INVENTION

1. Technical Field

The present invention relates to the field of treatment devices, and more particularly, to treatment devices and indications provided by the devices during surgical procedures.

2. Discussion of Related Art

Various types of information can aid the performer of a treatment procedure to achieve an optimal result. Such information may regard the patient, the treatment tool and the treatment efficiency. In one example, the state of the tip of the treatment tool determines in some treatments the treatment efficiency.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a device comprising a treatment tool arranged to apply a treatment to a tissue, a control unit arranged to obtain or measure at least one parameter, and at least one marking element associated with the treatment tool and arranged to illuminate the tissue with a signal relating to the at least one obtained or measured parameter and generated by the control unit.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A-1G are high level schematic illustrations of a treatment device, according to some embodiments of the invention.

FIGS. 2A and 2B are high level schematic illustrations of a single fiber device and a multi-fiber device, respectively, according to some embodiments of the invention.

FIG. 3A-3D are high level schematic illustrations of glove and hand associated devices, respectively, according to some embodiments of the invention.

FIG. 4 is a high level schematic flowchart illustrating a method, according to some embodiments of the invention.

FIGS. 5A, 5B, 6A-6C and 7A-7D are high level schematic illustrations of devices for cleaning and/or regenerating the treatment tip, according to some embodiments of the invention.

FIG. 8 is a high level schematic flowchart illustrating a tip regeneration method, according to some embodiments of the invention.

FIGS. 9A-9E, 10A, 10B, 11A and 11B are high level schematic illustrations of a device for controlling fiber bending, according to some embodiments of the invention.

FIGS. 12A and 12B are high level schematic illustrations of safety assemblies, according to some embodiments of the invention.

FIG. 13A is a high level schematic block diagram of a cordless surgical laser unit, according to some embodiments of the invention.

FIG. 13B is a high level schematic block diagram of treatment devices, according to some embodiments of the invention.

FIG. 14 is a high level schematic illustration of a welding-promoting device, according to some embodiments of the invention.

FIGS. 15A-15F are high level schematic illustrations of MCVD (modified chemical vapor deposition) fiber production methods, according to some embodiments of the invention.

FIGS. 16A-16B are high level schematic illustrations of optical fibers produced from coreless coated fibers, according to some embodiments of the invention.

FIG. 16C is a high level schematic illustration of optical fibers produced by outside vapor deposition (OVD) fabrication method, according to some embodiments of the invention.

FIG. 17 is a high level schematic illustration of a sheet of woven optical fibers, according to some embodiments of the invention.

FIG. 18 is a high level schematic flowchart illustrating a method, according to some embodiments of the invention.

### DETAILED DESCRIPTION OF THE INVENTION

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

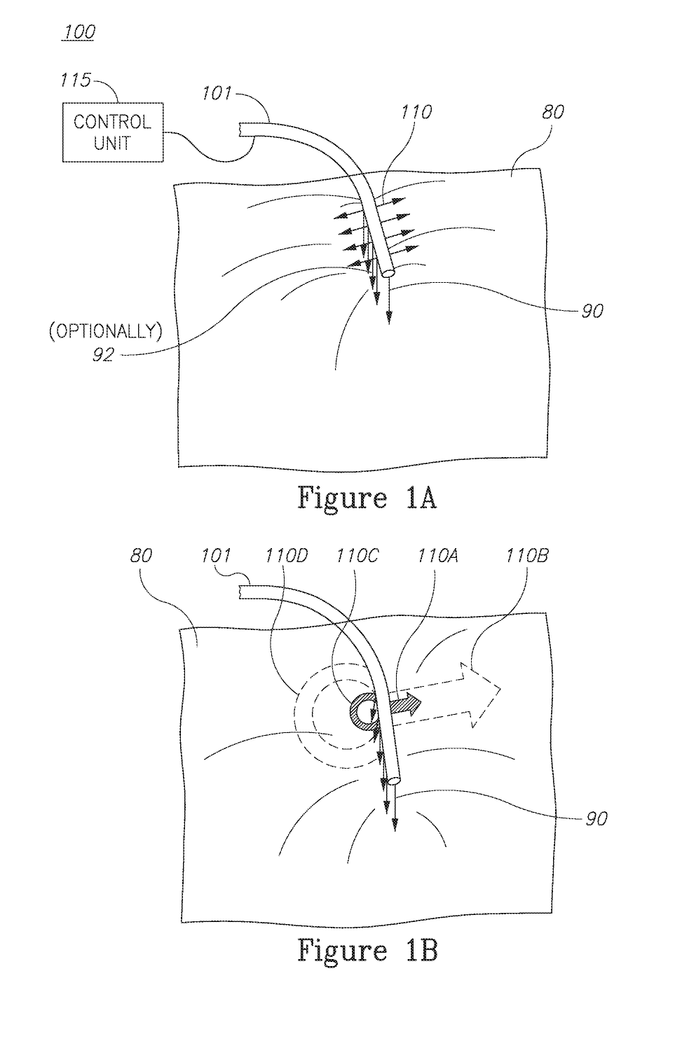

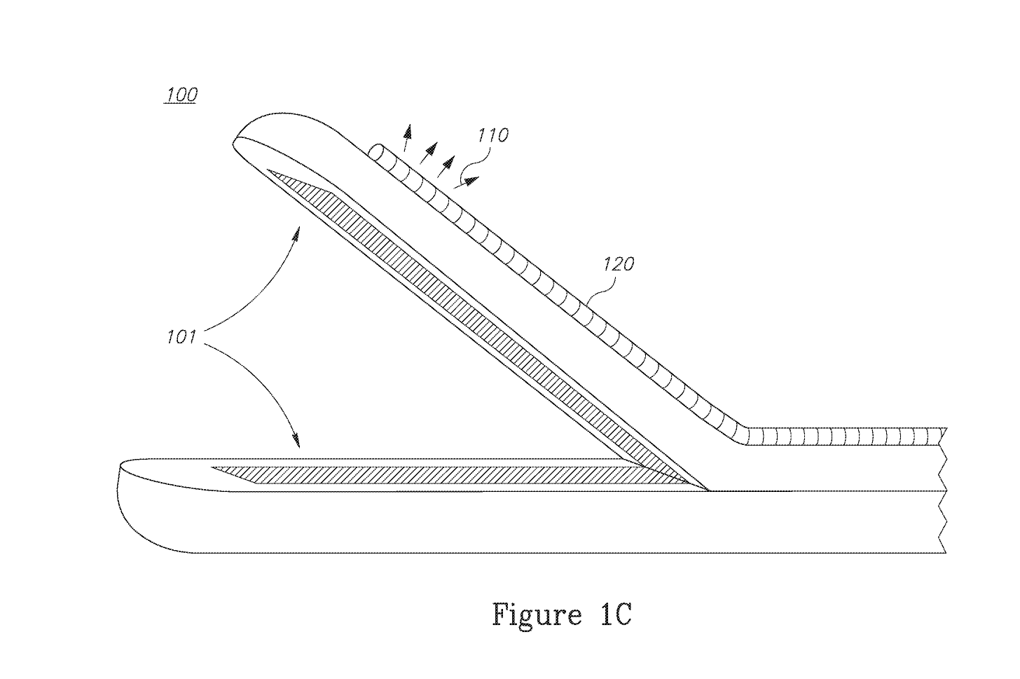

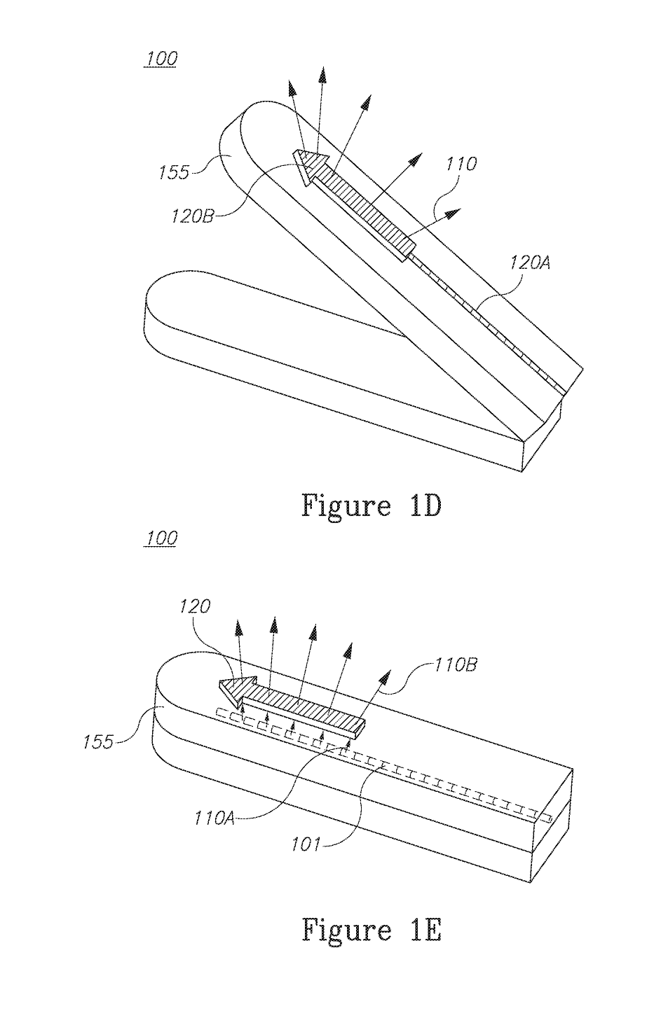

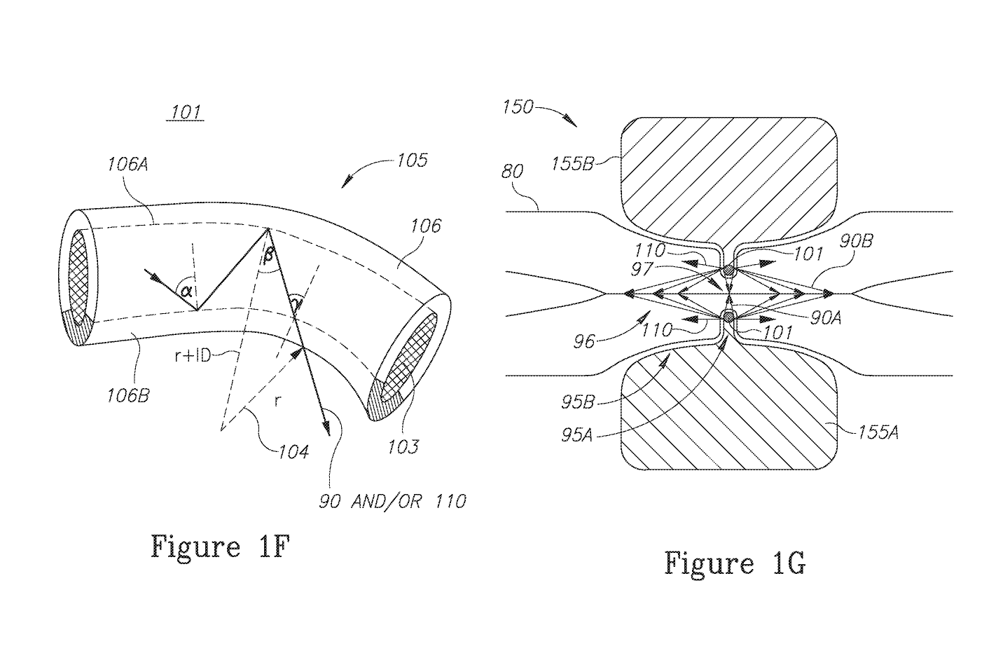

FIGS. 1A-1G are high level schematic illustrations of a treatment device 100, according to some embodiments of the invention.

Device 100 comprises a treatment tool 101 arranged to apply a treatment (in a non-limiting example, radiation 90) to a tissue 80, a control unit 115 arranged to measure or import at least one parameter, e.g., parameters indicative of device functioning, patient status or the efficiency of the treatment, and at least one marking element 120 (see below) associated with treatment tool 101 and arranged to illuminate the tissue with a visual indication signal 110 relating to the at least one obtained or measured parameter and generated by control unit 115. In certain embodiments, treatment tool 101 may be arranged to emit a sensing signal 92 to measure tissue parameters (e.g., optical parameters such as index of refraction or radiation absorption, electrical parameters such as impedance, mechanical parameters, flow parameters or temperature).

In certain embodiments, treatment tool 101 may comprise any of a passive treatment tool (e.g., a scalpel), an electric treatment tool (e.g., a RF delivering device), a laser treatment tool and/or an ultrasonic treatment tool. In certain embodiments, at least one marking element 120 may comprise at least one light emitting diode (LED), positioned, for example, on a tip of treatment tool 101. In certain embodiments, control unit 115 may be further arranged to generate signal 110 with respect to a treatment type applied by treatment tool 101 (e.g., tissue welding, tissue cutting). Control unit 115 may be further arranged to generate signal 110 with respect to obtained or measured parameters (e.g., measured by sensing signal 92 or obtained from an external measurement device such a patient monitor) which are applicable to each of the treatment types. In certain embodiments, marking element 120 may be arranged to illuminate the treated tissue or its surrounding by signal 110 and/or by providing additional illumination.

Device 100 is arranged to deliver visual indication 110 directly on tissue 80, signal 110 may be configured to encode specific information that is related to the treatment process. For example, a progress of tissue sealing or cutting processes may be color coded. Advantageously, providing the indication in proximity to the treated area may increase its cognitive assimilation by the treating physician or surgeon. It is noted that in modern day surgical operations, the physician is fed with large amounts of information from numerous sources which relay the patient's status, feedback on the performance of various instruments in use and much more. During surgery the physician or surgeon is focused on the operable field (including tissue 80), observing the screen and looking for any changes in color and texture of tissue 80 as well as for bleeding. Advantageously with respect to products which produce audible indicators, device 100 directly illuminates tissue 80 (or parts of device 100 itself) and thus avoids potentially disturbing and ambiguous sound cues.

For example, signal 110 may be a red or green illumination on surrounding tissue, indicating the end of a cutting process and of a sealing process, respectively (or vice versa). In certain embodiments, different colors or intensities may indicate parameters relating to each process by itself. For example, the illumination intensity may be related to the advancement of the process and to its efficiency. In certain embodiments, additional information on the process may be provided by additional audible signals.

In certain embodiments, signal 100 may relate to the obtained or measured parameter(s) by any of a signal color, a signal intensity, a signal periodicity, pattern size and/or a direction of the signal.

FIG. 1B for example, schematically illustrates two types of possible signals, according to some embodiments of the invention. Signals 110A and 110B schematically illustrate an animated indication of direction, exhibiting a temporal change of signal 110 which may be configured to relate to the at least one obtained or measured parameter by a temporal change of the signal (e.g., the width, length and color of the arrow may change with respect to any of the parameters that are displayed by signal 110). Signals 110C and 110D schematically illustrate another type of signal changes, namely changes in extent and possibly pattern, as well as a changing region of illuminations. These signal changes may be configured to relate to any relevant obtained or measured parameter (e.g., as measured by sensing signal 92 and/or obtained from external instruments). Generally, signal 110 may be configured to exhibit a temporal change that creates dynamic illumination of tissue 80. In certain embodiments, illumination 110 of tissue 80 relates to the at least one obtained or measured parameter by any of an extent of illumination 110, an intensity of illumination 110, a color of illumination 110, frequency of illumination 110 and a region (on tissue 80 and/or on device 100) of illumination 110.

Advantageously, device 100 provides the physician or surgeon a direct feedback on the progress of, e.g., cutting, sealing or soldering tissue and/or blood vessels, and allows the surgeon to directly observe the progress of the process. Providing respective signals 110 is the most direct way to provide such feedback and within the operable field, where the surgeon's attention is. Signal 110 may be continuous or exhibit short pulses which do not obscure or disturb the operative field. In certain embodiments, signal 110 in different colors may be used to convey different meanings or different sequences of pulses or patterns. For example, signal 110 may indicate both the type of parameter (e.g., tissue temperature, index of refraction or impedance status) as well as the values of the obtained or measured parameters in different modalities (e.g., color and tempo-spatial pattern, see FIG. 1B). In addition, signal 110 may also contain information relating to treatment assessment (e.g., progress of a sealing or cutting process). Signal 110 may be continuous or may be dynamic and include a certain animation to capture the dynamic nature of the process.

In certain embodiments, the at least one obtained or measured parameter may comprise any of the tissue parameters, the device parameters and the patient parameters. Obtained or measured tissue parameters may comprise, for example, tissue temperature, the index of refraction at a tissue interface, tissue impedance and Doppler shift measurements of the tissue or of tissue fluids (measured e.g., by sensing signal 92 or obtained from external devices). Examples for measurements may comprise optical parameters related to the scene of treatment, such as index of refraction or impedance which may be used to derive tissue temperature and tissue status. In some embodiments, Doppler shift measurements may be used to assess the seal quality by measuring minute changes in flow speeds. Device parameters may comprise, for example, operational parameters of device 100, indication of device failure and indication of device readiness. Patient parameters may comprise, for example, patient's blood pressure and patient's physiological information. Device and patient parameters may be measured and/or obtained from an external source.

FIG. 1C schematically illustrates device 100 comprising treatment tool 101 having a treatment zone (hatched) which may operate according to any treatment method (electrically, by ultrasound, by laser, mechanically, by heat etc.) and having marking element 120 (in this non-limiting example, an optical fiber) configured to emit signal 110.

FIG. 1D schematically illustrates device 100 comprising a treatment tool such as surgical forceps having a jaw 155 with a marking element. The marking element may comprise a fiber 120A ending with a member 120E arranged to diffuse electromagnetic radiation delivered by fiber 120A and produce signal 110. For example, member 120B may be filled with a light diffusing material. The marking element may be implemented to any treatment tool, which may operate according to any treatment method (electrically, by ultrasound, by laser, mechanically, by heat etc.).

FIG. 1E schematically illustrates device 100 comprising treatment tool 101 such as optical fiber 101, associated in the illustrated example with surgical forceps. Fiber 101 may be arranged to emit radiation 110A as a preliminary signal, which amplified, modified and/or redirected by marking element 120 to produce signal 100B that indicates parameters as described above and/or illumination of the treatment region. For example, radiation 110A may be infrared and marking element 120 may comprise a material which glows when illuminated by infrared radiation 110A. In another example, marking element 120 may comprise a temperature sensitive material which emits radiation which is related to its temperature. Marking element 120 may be implemented to any treatment tool, which may operate according to any treatment method (electrically, by ultrasound, by laser, mechanically, by heat etc.).

FIG. 1F is a high level schematic block diagram of an emitting region of an optical fiber 101, according to some embodiments of the invention. Optical fiber 101 comprises a core 103 having a refractive index n_{K} and a cladding 106 having a refractive index n_{M}. Optical fiber 101 has at least one specified region of cladding 106 that is arranged to emit electromagnetic radiation from core 103 upon bending optical fiber 101 at the specified region beyond a specified bending threshold with respect to a bending radius r 104. The emission of electromagnetic radiation from core 103 upon bending optical fiber 101 is related to in the following as bend-emission (BE). The bend emission depends on various fiber and radiation characteristics such as the size, structure and materials of the fiber, bending radius 104, radiation frequency and so forth. The fiber is designed to enable bend emission in the specified region and sectors only, while continuing to prevent transmission through the cladding in other parts of the fiber.

It is noted that bend-emission may be configured to occur inwards or outwards with respect to the direction of bending. It is further noted that the disclosed principles are also applicable to other types of waveguides, e.g., RF waveguide, which may be tailored for specific geometrical parameters allowing highly controlled and specific emission patterns.

Bend emission may be achieved by bending fiber 101 prior to an actual application thereof, e.g. bending fiber 101 to have a snare-like form, and angled form, a stent-like form etc. (see examples below), and then controlling the bend emission by the light source upon placing the bended regions of fiber 101 in an operative position. Alternatively or complementary, bend emission may be under geometrical control, achieved by making use of the natural curvature of the targeted object to generate the desirable energy discharge profile from the waveguide. Certain regions in fiber 101 may be designed to bend-emit upon curving in contact with the target, as exemplified below, and the energy that is emitted in bends in these regions is actually used to achieve the desired goal. In such case, emission may be controlled by the actual bending, in addition or in place of controlling the light source.

Any type of fiber 101 may be arranged to emit radiation upon a specific bending, e.g. a waveguide (which may comprise metallic waveguides), a solid core optical fiber, a hollow fiber and a photonic crystal fiber (such as a holey fiber, a Bragg fiber or any other micro-structured fiber). The non-emitting sector(s) may be micro-structured (e.g. with a grating or air holes) to reduce an effective refractive index thereof below a refractive index of the emission sector and/or to direct radiation toward the emission sector.

It is noted that fibers 101 described throughout the disclosure may be round, elliptic, trapezoid or have any other cross section form. For example, fibers 101 may be flattened mechanically or be machined to have any cross sectional form, e.g., a round or elliptic form with machined flat sides. In certain embodiments, treatment fiber comprises at least one specified region 105 of a cladding thereof, also termed “emitting region” herein, that is arranged to emit electromagnetic radiation from a core thereof upon bending of fiber 101 at the at least one specified region 105 beyond a specified bending threshold. A cross section of at least a part of treatment fiber 101 may be flattened at the at least one specified region 105 to provide mechanical control of the placing of fiber 101 and for mechanically stabilizing the fiber bend. Moreover, the flattened side may be used to enhance fiber contact with the treated tissue and ease manipulating the emitting region to bend upon the treated tissue and thus enhance the treatment efficiency. The flattening may be achieved mechanically (e.g., by pressing fiber 101 or its preform), by machining the preform or the fiber or by manipulating the drawing stage in the manufacturing procedure to yield a specified cross section. Specifically, any of the fiber embodiments may have a cross section which is radially asymmetric, e.g., flattened, elliptic, polygonal, truncated etc.

Optical fiber 101 may be single-mode or multi-mode, in the latter case, the specified emission region and bending threshold may be selected with respect to the required modes, to control the emitted energy. In addition, the specified emission region and bending threshold may be selected with respect to, and controlled by, the beam polarization.

Bends in fiber 101 that may be used in emitting regions 105 include both micro-bends (local deviations from the fiber's linearity, with relative small bending radii) and macro-bends (changes of angle of the fiber's direction, usually larger bending radii). For example, the emitted radiation from a macro-bend may be estimated, for single mode fibers, by the expression: Exp (8.5−519·D·(2λ·MFR))³) in dB/m, where D is the bending radius in mm, λ is the wavelength in λ_{ce} is the fiber cut-off wavelength in μm and MFR is the mode fiber radius in μm.

FIG. 1F illustrates the condition for bend emission, according to some embodiments of the invention, by illustrating an example of a possible trajectory of light travelling down fiber 101. The light reaches the beginning of the bended specified region 105 at an angle α that is not yet sufficient for BE due to the bend radius which is not sufficiently small at this point (i.e. α is still larger than θ_{emitting region}=sin⁻¹(n_{E}/n_{K}). The light is thus reflected to cladding side 106A and is internally reflected at angle β which is larger than θ_{cladding}=sin⁻¹(n_{M}/n_{K}), and stays within core 103. Upon reflection from cladding 106A the light reaches sector 105 with cladding 106B at an angle γ which is now smaller than θ_{emitting region}=sin⁻¹ (n_{E}/n_{K}), as the bending radius reaches at this point the threshold radius and goes beyond a specified bending threshold of the specified region of fiber 101 (i.e. bending radius becomes smaller than the threshold radius). The transmitted light exits region 105 at an angle θ. The exact calculation follows Wang et al. (2007) “Investigation of Macrobending Losses of Standard Single Mode Fiber with Small Bend Radii”, in Microwave and optical letters 49:9, 2133-2138. Bend-emission can be approximated as starting to occur at angle θ_{bend} that is defined using bending radius r 104 as: θ_{bend}=sin⁻¹(r_{bend}/(r_{bend}+ID)), ID being the internal diameter i.e. core diameter. The condition for bend-emission is that sin⁻¹(n_{M}/n_{K})=θ_{cladding<}θ_{bend}<θ_{emitting region}=sin⁻¹(n_{E}/n_{K}).

Other than the prior art, the present invention utilizes conditional and controllable side emissions from an optical fiber. In contrast to side firing fibers, fibers of the present invention do not emit any radiation when straight or bended below the bending threshold. The side emission is activated only upon the bending of the fiber at a predetermined bending radius, for example by an obstruction that is to be removed by the fiber, or according to a specific device design.

During treatment by emitted radiation 90 (or 110, i.e., either or both treatment and marking radiation may be emitted by bending fiber 101), parts of the treated target (e.g., a flow obstruction or a polyp) are removed, causing the target to be decimated and flattened. In some embodiments, target flattening reduced the bending of fiber 101 (increases the bending radius thereof) and causes a reduction in bend emission until conclusion of the treatment. Such effect may be desired and taken into account when selecting the bending threshold. In some embodiments, a different specified region may take over the treatment, and be activated by a different bending threshold to allow multi-stage treatment.

FIG. 1G is a high level schematic illustration of surgical forceps 150 having a vessel sealing tip, according to some embodiments of the invention. The vessel sealing tip may comprise an energy delivery element 101 such as at least one optical element 101 arranged to deliver, upon actuation, electromagnetic radiation 90 to a vessel 80 to cut vessel 80 at a cutting region or to yield a vessel welding effect in a specified sealing section 96 of vessel 80, and to cut vessel 80 at a cutting location 97 within specified sealing section 96. For example, at least one optical element 101 may comprise at least one optical fiber 101 arranged to deliver electromagnetic radiation such as laser energy, for example in different angles, intensities or wavelengths 90A, 90B, e.g., to apply different types of treatment (such as vessel cutting and vessel sealing, respectively). In certain embodiments, the tip may emit signal 110 onto the treated vessel or laterally from the treatment location. Energy delivery element 101 may be attached to any one of two jaws 155 (155A, 155B) of forceps tip 150, or may also be a free element, at least on a part of the length thereof. In certain embodiments, device 150 may comprise a sealing and dissection grasper tool with at least two different mechanical pressure levels for sealing and/or cutting.

In certain embodiments, at least one jaw 155 of the forceps may comprise at least one protrusion 95A arranged to constrict vessel 80 prior to the actuation of energy delivery element 101 (such as at least one optical element 101, a RF source, an ultrasound source etc.). Protrusion 95A protrudes from a surface 95B of jaw 155 and constricts vessel 80 at the region of energy deliver to reduce the local thickness of vessel 80 and to provide more spatial variability in possible energy delivery directions. Energy delivery element 101 may be positioned fully or partially within protrusion 95A; for example, at least one optical element 101 may be set within at least one protrusion 95A.

Certain embodiments of the invention comprise a tip 100 with at least two jaws 155 for surgical forceps 150. At least one of jaws 155 may comprise at least one protrusion 95A positioned to contact tissue held by the tip and deliver both pressure and external energy to the tissue. The pressure may be a tip holding force (the force applied to the forceps and thereby transferred to the tip's jaws), concentrated by at least one protrusion 95A. The external energy may be any of electromagnetic (e.g., optical, RF), electrical and ultrasound energy, or a combination thereof. At least one protrusion 95A may comprise one or more thin element that concentrates applied forces onto a small section of vessel 80. At least one protrusion 95A may comprise an abrasive or an ablative element that reduces vessel wall thickness or even cuts the vessel, in addition to constricting the vessel.

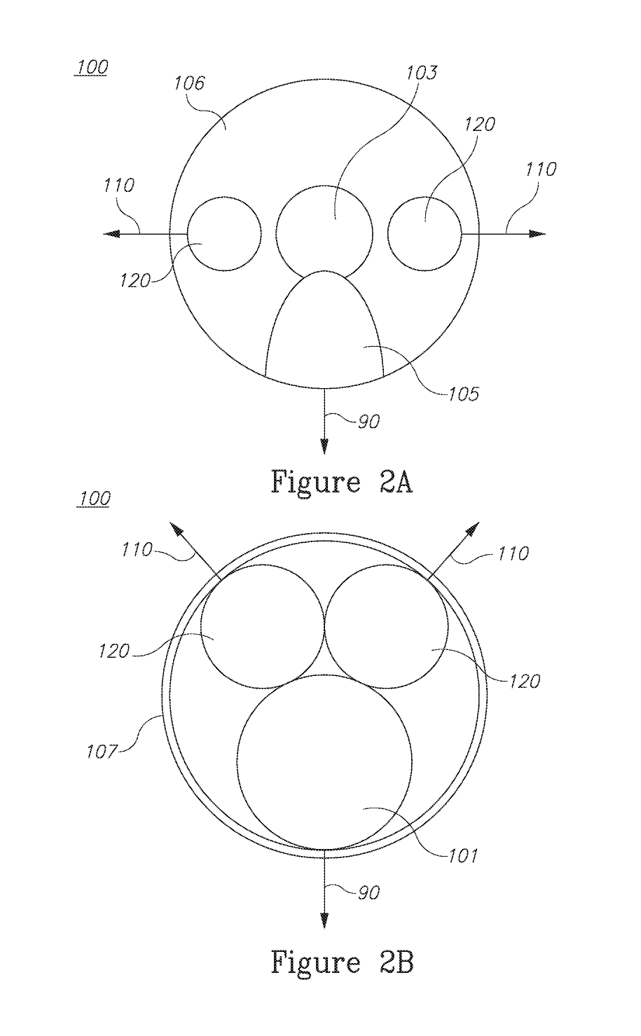

In certain embodiments, treatment tool 101 may comprise at least one treatment fiber 101. FIGS. 2A and 2B are high level schematic illustrations of single fiber device 100 and multi-fiber device 100, respectively, according to some embodiments of the invention. In certain embodiments, at least one marking element 120 may comprise at least one marking core 120 (FIG. 2A) which is implemented together with at least one treatment fiber 101 as a single fiber 100 and/or at least one marking fiber 120 (FIG. 2B) associated with treatment fiber 101.

In certain embodiments, illustrated in a non-limiting example in FIG. 2A, treatment fiber 101 may comprise at least one specified region 105 of a cladding 106 thereof, which is arranged to emit electromagnetic radiation 90 from a core 103 thereof upon bending of fiber 101 at at least one specified region 105 beyond a specified bending threshold. Fiber 101 may further comprise at least one marking core 120 arranged to emit signal 110.

In certain embodiments, treatment fiber(s) 101 is configured to apply at least two types of treatment to tissue 80 and control unit 115 may be arranged to generate signal 110 with respect to the treatment type applied by treatment fiber(s) 101.

In certain embodiments, illustrated in a non-limiting example in FIG. 2B, marking fiber(s) 120 may be within a jacket 107 of treatment fiber 101, or may be attached to any type of treatment tool 101. In certain embodiments, at least one marking fiber 120 and at least one treatment fiber 101 may be implemented as respective cores (see, e.g., FIG. 2A) with single fiber device 100. For example, main core 103 may deliver energy as therapeutic means while additional cores 120 may deliver energy to generate illumination signal 110, to relay information of the operator.

In any of the configurations, marking fiber 120 may be arranged to illuminate tissue 80 sideways with respect to a direction of treatment application 90 by treatment fiber 101 (see FIGS. 1A, 1B, 2A, 2B).

Signal emission may be carried out e.g., by direct emission and/or scattering at specified regions 120 of treatment tool 101 such as fiber 101. In particular, signal emission may be carried out by fiber cores 120. Specific cores 120 may be implemented only at certain regions of fiber 101, for example close to the fiber tip or at a specific pattern along fiber 101. In certain embodiment signal emission may be carried out by areas of fiber cladding 106 and/or jacket 107. The extent of signal emission along treatment tool 101 may be configured according to specified used of treatment tool 101.

In certain embodiments, signal emission may be carried out upon bending of respective tool regions beyond specified thresholds. For example, in case of treatment fiber 101, the refractive indices of cores 120 and/or cladding 106 may be arranged to enable some of the illumination going through the fiber to escape and thus be used as signal 110.

In certain embodiments, device 100 may comprise treatment tool 101 and marking element(s) 120 (without control unit 115). Treatment tool 101 and/or marking element(s) 120 may be arranged to directly measure at least one parameter and marking element(s) 120 may be arranged to illuminate the tissue with signal 110 relating to the at least one obtained or measured parameter. For example, marking element(s) 120 may be arranged to couple changes in the index of refraction of the tissue with signal parameters to generate thereby treatment indications. For example, treatment fiber 101 may be configured in a way that causes certain values of the index of refraction to change the emission characteristics of fiber 101 to emit signal 110.

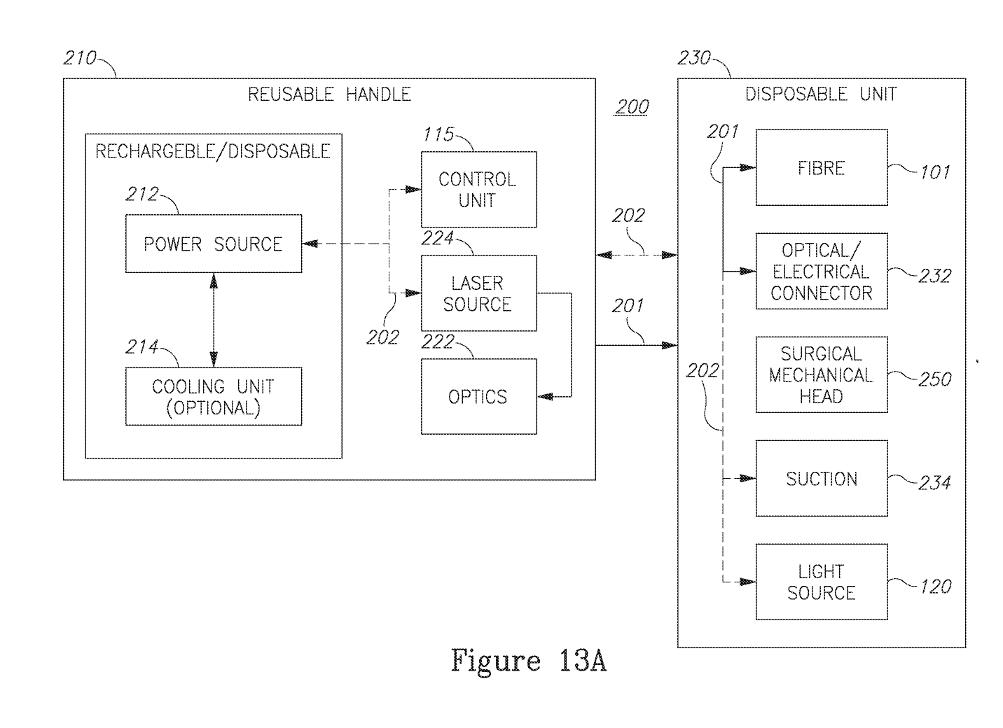

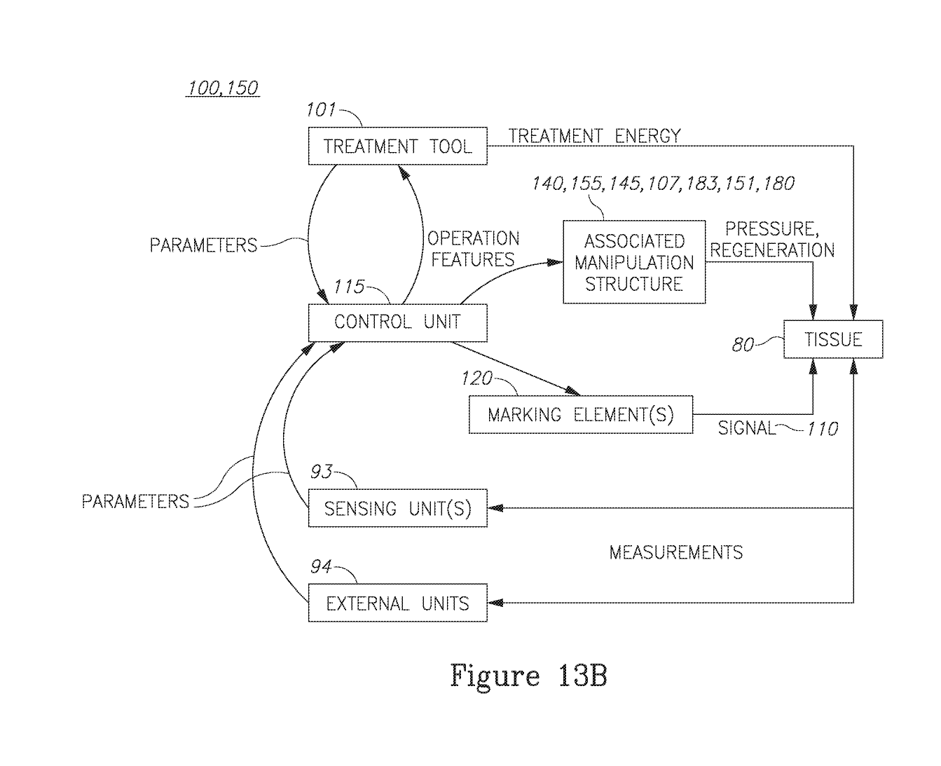

In certain embodiments, parameter measurements may be carried out optically by treatment tool 101 and/or by marking element(s) 120. For example, in treatment fiber 101, the intensity of reflected treatment radiation may be used to derive tissue or treatment related information as well as patient related information (e.g., pulse or oxygen saturation). In certain embodiments, by marking element(s) 120 such as fiber 120 and/or core 120 may be used to optically measure parameters in the scene of treatment. In certain embodiments, a sensor 93 (see FIG. 13B) may be used to measure at least one parameter and deliver the obtained or measured parameters to control unit 115 and/or to marking element(s) 120. In certain embodiments, control unit 115 and/or the sensor may be implemented at the tip of treatment tool 101, for example at the tip of fiber 101.

In certain embodiments, treatment tool or fiber 101 comprises at least one side firing fiber as marking element 120.

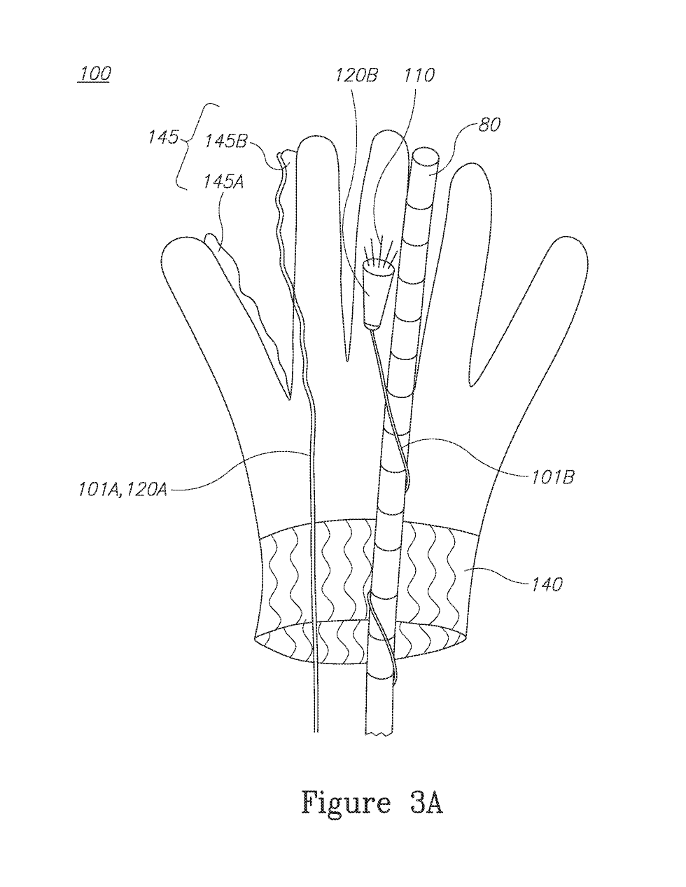

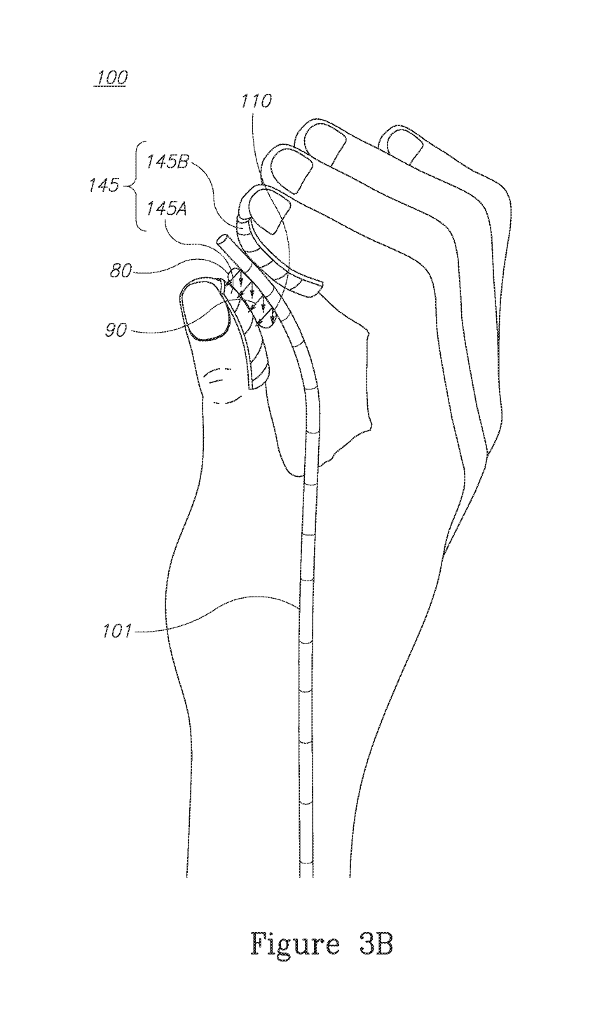

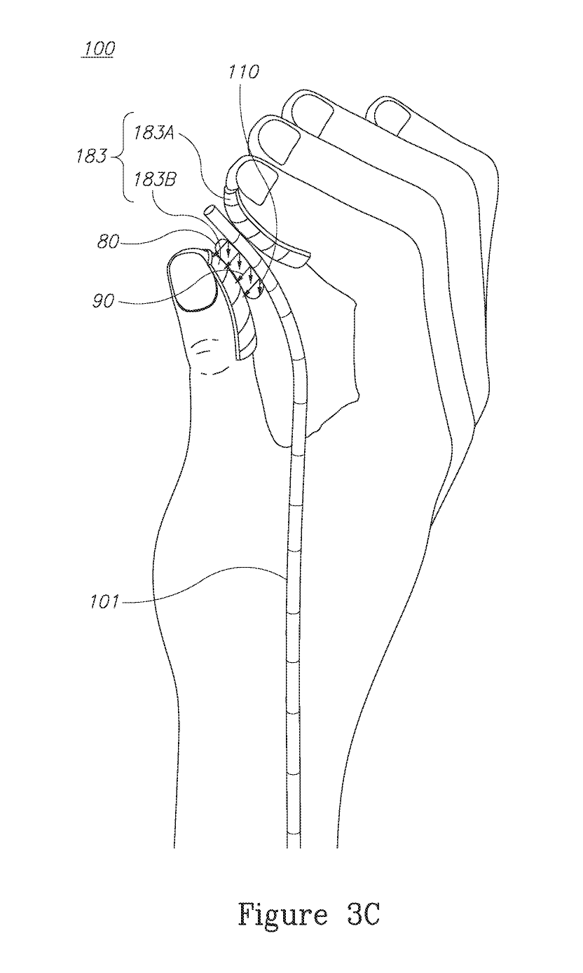

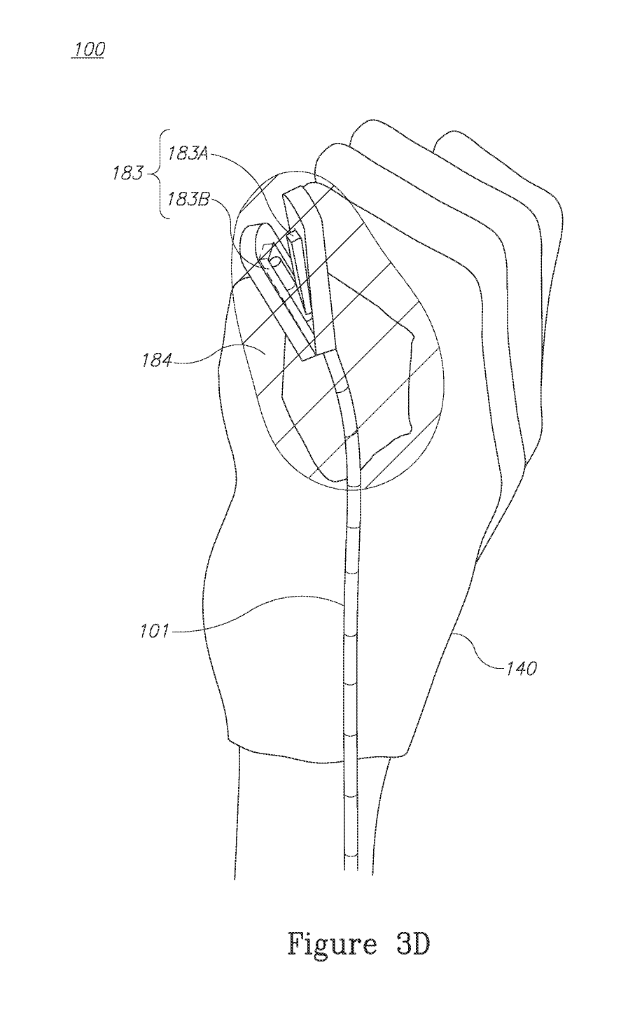

FIG. 3A-3D are high level schematic illustrations of glove and hand associated devices 100, respectively, according to some embodiments of the invention. Treatment tool 101 and/or marking element 120 may be associated with a treatment glove 140 or be configured to allow efficient manipulation by hand. Treatment gloves 140 and hand associated devices 100 may be implemented to have any, all or none of the features described in the present disclosure independently of other features, for example, treatment gloves 140 and hand associated devices 100 may be implemented only with treatment tool 101, only with marking element 120 or with of their combinations presented in the disclosure.

Glove-associated device 100 may comprise glove 140 onto which treatment tool 101 is mounted. In certain embodiments, treatment tool 101B may be a fiber or any other treatment tool, and marking element 120B may be associated with treatment tool 101B, e.g., wound around it or attached at its end (marking element 120B is a LED example). Marking element 120B may be, for example, a fiber or a LED emitting signal 110. In certain embodiments, treatment tool 101A may be a fiber 101A with or without marking fiber 120A (see exemplary embodiments in FIGS. 1A, 1B, 2A, 2B). In the example illustrated to by fiber 101B in FIG. 3A, fiber 101B may be wound around a blood vessel as tissue 80 in order to seal and cut vessel 80. LED 120B may be used to indicate flow in vessel 80, a treatment effect on vessel 80, physiological parameters such as blood pressure or pulse as measured on vessel 80, clinical status of the patient and/or a treatment mode of device 100 such as sealing or cutting operation mode etc. In certain examples, measurements, e.g. of blood pressure, may be used to control the treatment mode, e.g. identify the end of necessary sealing treatment before application of a cutting treatment.

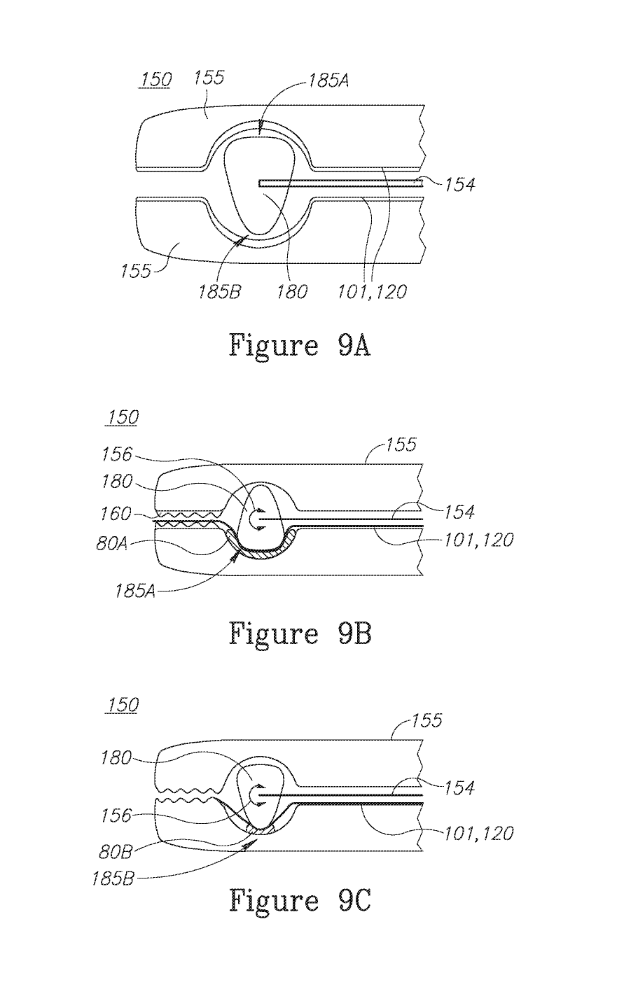

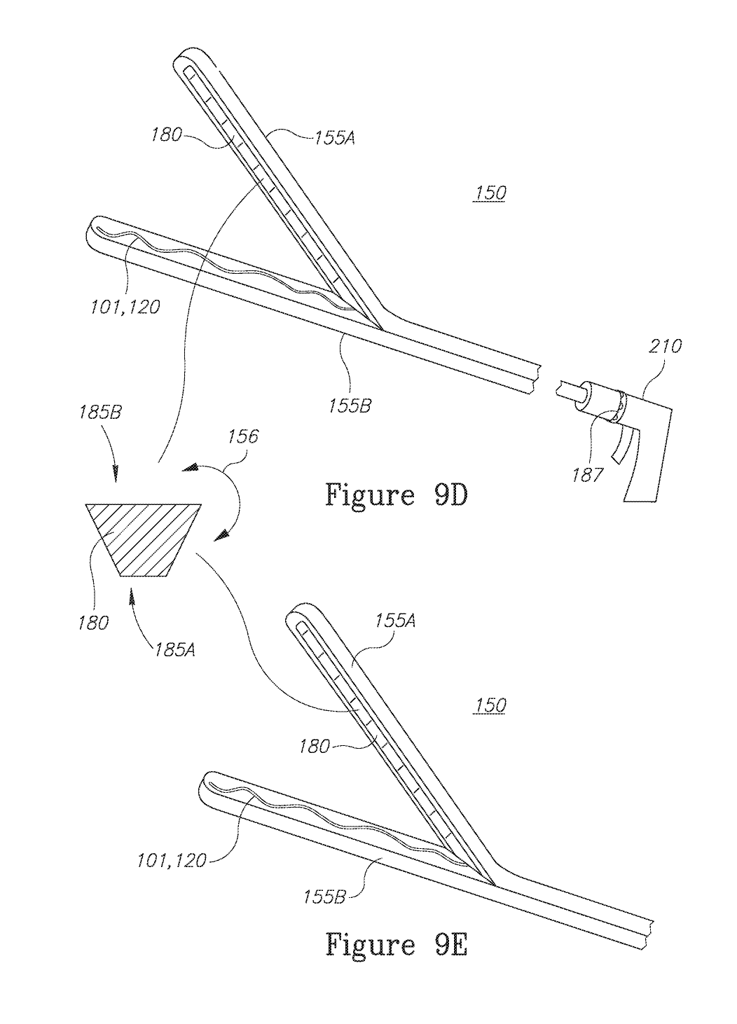

Fiber 101A may be manipulated by hand with or without a placing template 145. For example, such template 145 may comprise two supports 145A, 145B configured to facilitate treatment tool application to the tissue, for example by supporting fiber 101A and/or treated tissue 80 to apply the treatment correctly. In certain embodiments, supports 145A, 145B may be configured as jaws for holding tissue 80 and/or fiber 101A (see below, FIGS. 9A-9E, 10A, 10B, 11A and 11B for non-limiting jaw examples). In certain embodiments, fiber 101A and/or marking fiber 120A may be woven into glove 140 or interwoven therewithin. Hand-associated device 100 may similarly comprise fiber 101 for treatment and/or marking, arranged to be hand-held (without glove 140). Supports 145A, 145B may be provided to improve tissue and fiber handling. Such applications may utilize the marking signals to enhance the surgeon's treatment dexterity.

In certain embodiments, wherein treatment tool 101 is treatment fiber 101 configured to emit radiation from bended regions of fiber 101, device 100 may be configured as hand held device 100 (FIG. 3B) in which fiber 101 is bended manually. Emission may be controlled by the surgeon's fingers, bending fibers 101 and/or 120 to specified ranges of radii of curvature, which may be personalized according to the surgeon's preference and type of treatment.

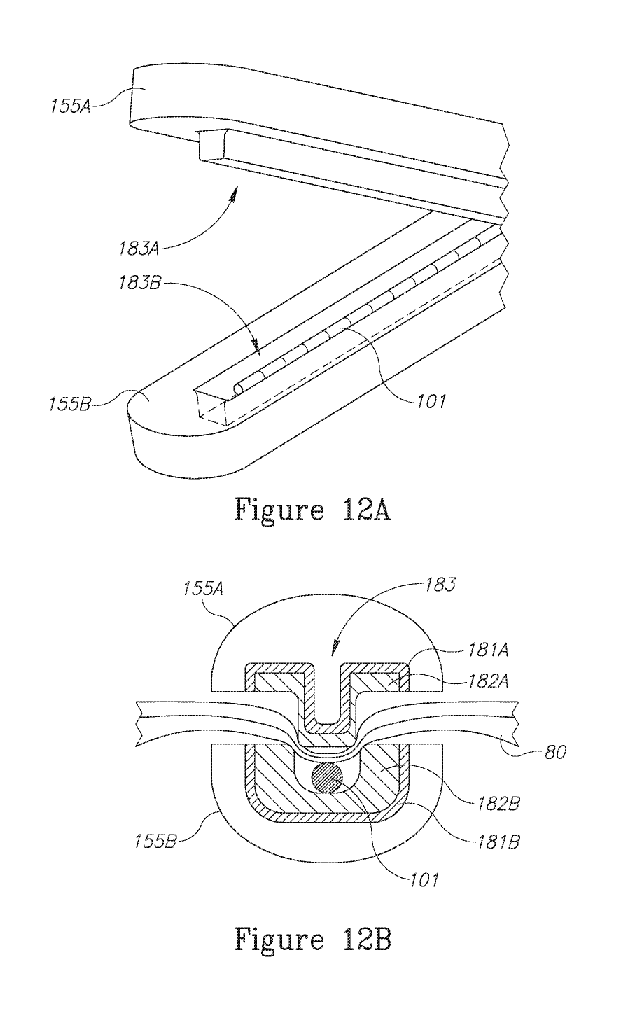

FIG. 3C schematically illustrates a hand associated device 100 which may comprise a safety assembly 183 having supports 183A, 183B arranged to protect the practitioner from laser radiation and to ensure the treatment efficiency. Supports 183A, 183B may be implemented in a similar way to safety assembly 183 illustrated in FIGS. 12A and 12B described below. Certain embodiments comprise treatment gloves 140 having safety assembly 183 as illustrated in FIG. 3D. Treatment gloves 140 and hand associated devices 100 may further comprise a protective shield 184 for filtering potentially harmful radiation such as emitted laser radiation, and ensuring compliance with laser eye safety regulations. Any of the elements illustrated in FIGS. 3A-3D may be implemented in any configuration of device 100, independently of other illustrated elements.

In certain embodiments, the pressure applied either via glove 140 or by the surgeon's hand may be used to determine the treatment type, e.g., relatively low pressure may be used to apply a sealing treatment while a higher pressure may be used to apply a cutting treatment. The connection between pressure application and treatment type may be direct and mechanical, may be controlled by the degree of fiber bending (e.g., by configuring the bending thresholds of the fiber), may be controlled using measurements from a sensing element (e.g., via sensing signal 92) or the fiber itself, and/or be controlled in an open loop via signals 110 emitted by marking element(s) 120.

In certain embodiments, device 100 comprises treatment tool 101 arranged to apply a treatment to tissue 80, and treatment tool 101 is arranged to be finger-held and to apply the treatment upon finger manipulation of tool 101. Device 100 may further comprise a safety member arranged to protect a practitioner from being damaged by tool 101. For example, the safety member may comprise protective shield 184. In case of treatment tool 101 being a laser, protective shield 184 may be configured to provide specified laser eye safety requirements. In certain embodiments, the safety member may comprise two complementary supports 183A, 183B arranged to enclose treatment tool 101 and treated tissue, for example, when treatment tool 101 is a laser, complementary supports 183A, 183B may enclose a treatment section of the laser and a treated tissue section. In certain embodiments, any of devices 100 described above may be associated with treatment glove 140 to which the safety member is connected.

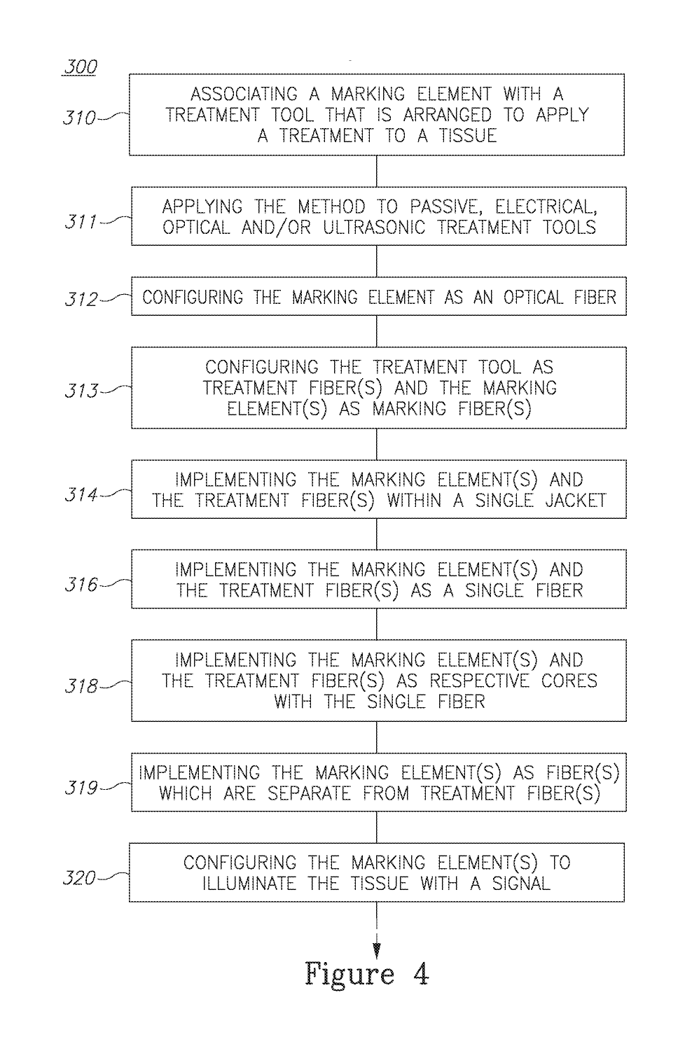

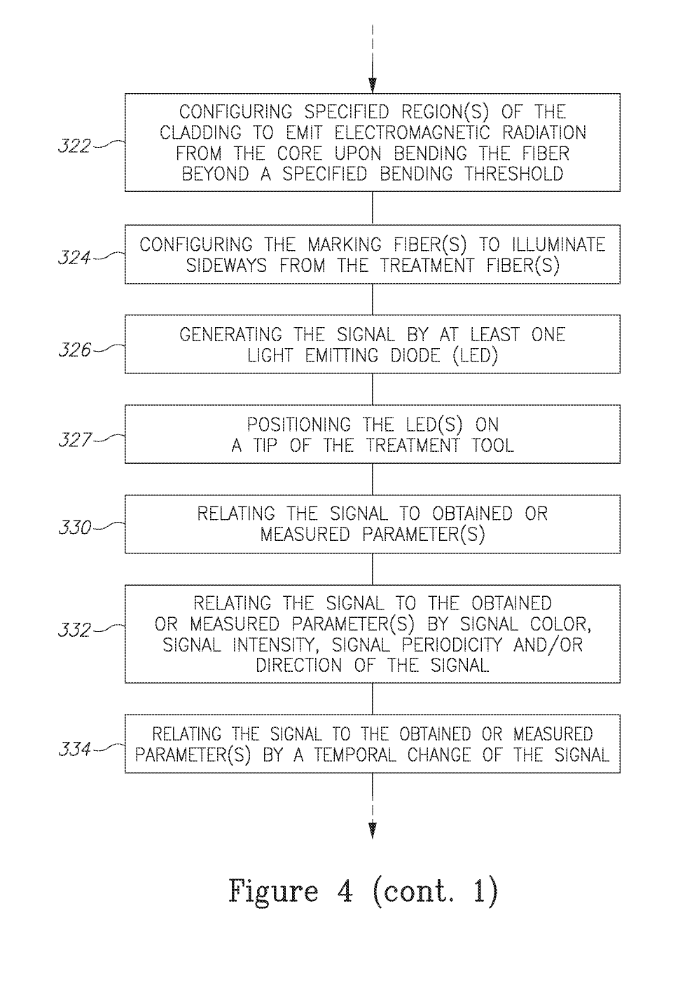

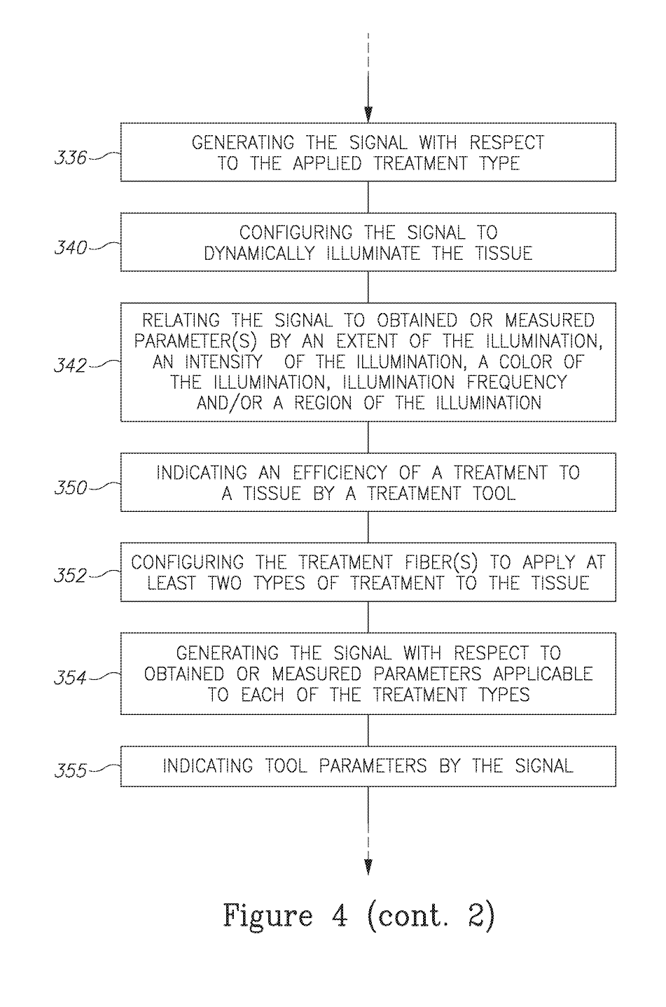

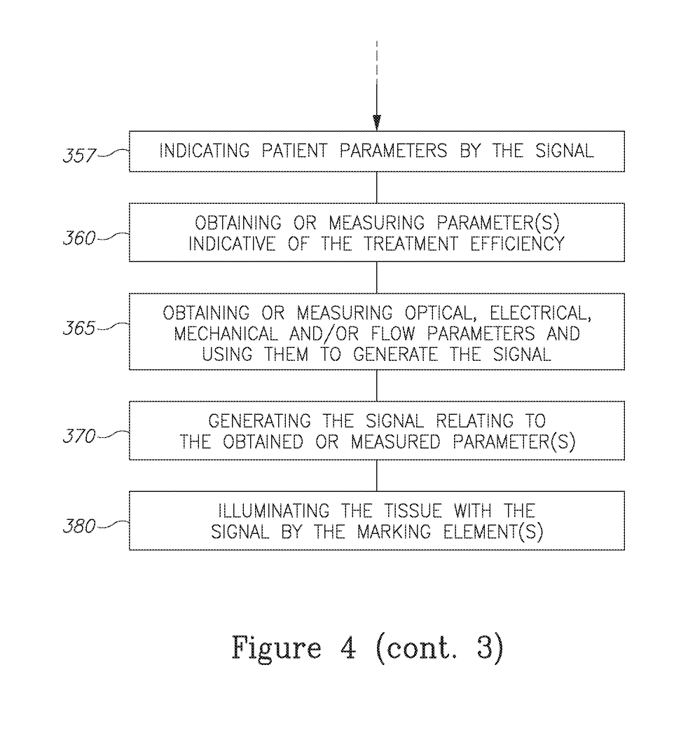

FIG. 4 is a high level schematic flowchart illustrating a method 300, according to some embodiments of the invention. Method 300 comprises associating at least one marking element with a treatment tool that is arranged to apply a treatment to a tissue (stage 310) and configuring the at least one marking element to illuminate the tissue with a signal (stage 320). In certain embodiments, method 300 comprises relating the signal to at least one obtained or measured parameter, e.g., parameters related to the treatment (stage 330). Method 300 may be applied to passive, electrical, optical and/or ultrasonic treatment tools (stage 311).

In certain embodiments, method 300 may comprise configuring the marking element as an optical fiber (stage 312) which may be attached to, associated with or independent from the treatment tool. The treatment tool may be a fiber, but may be any other treatment tool. In case the treatment tool is a fiber, the same fiber may be used to apply the treatment and implement the marking, treatment and marking fibers may be mechanically coupled or treatment and marking fibers may be independent from each other.

In certain embodiments, method 300 may comprise configuring the treatment tool as at least one treatment fiber and the at least one marking element as at least one marking fiber (stage 313). In certain embodiments, method 300 may further comprise implementing the at least one marking element and the at least one treatment fiber within a single jacket (stage 314). In certain embodiments, method 300 may further comprise implementing the at least one marking element and the at least one treatment fiber as a single fiber (stage 316). In certain embodiments, method 300 may further comprise implementing the at least one marking element and the at least one treatment fiber as respective cores with the single fiber (stage 318). In certain embodiments, method 300 may comprise implementing the marking element(s) as fiber(s) which are separate from treatment fiber(s) (stage 319).

In certain embodiments, method 300 may further comprise configuring at least one specified region of a cladding of the at least one treatment fiber to emit electromagnetic radiation from a core thereof upon bending of the fiber at the at least one specified region beyond a specified bending threshold (stage 322). Method 300 may further comprise configuring the at least one marking fiber to illuminate sideways from the at least one treatment fiber (stage 324). In certain embodiments, method 300 may further comprise generating the signal by at least one light emitting diode (LED) (stage 326) positioned, for example on a tip of the treatment tool (stage 327).

Relating the signal to obtained or measured parameters 330 may be carried out with respect to, for example, signal color, a signal intensity, a signal periodicity and/or a direction of the signal (stage 332). Relating the signal to obtained or measured parameters 330 may be carried out by a temporal change of the signal (stage 334). Method 300 may further comprise generating the signal with respect to a treatment type applied by the treatment tool (stage 336).

In certain embodiments, method 300 may further comprise configuring the signal to dynamically illuminate the tissue (stage 340). For example, configuration 340 may be carried out to relate to the at least one obtained or measured parameter by, for example, an extent of the illumination, an intensity of the illumination, a color of the illumination, and illumination frequency and/or a region of the illumination (stage 342).

In certain embodiments, method 300 may further comprise indicating an efficiency of a treatment to a tissue by a treatment tool (stage 350). Method 300 may comprise indicating tool parameters by the signal (stage 355) and/or indicating patient parameters by the signal (stage 357).

Method 300 may comprise measuring at least one parameter indicative of the treatment efficiency (stage 360). Method 300 may comprise measuring any of optical, electrical, mechanical and/or flow parameters and using the obtained or measured parameters to generate the signal (stage 365).

Method 300 may comprise generating a signal relating to the at least one obtained or measured parameter (stage 370), and illuminating the tissue with the signal by at least one marking element (stage 380). Method 300 may provide a signal relating to any of the obtained or measured parameters listed above.

In certain embodiments, method 300 may further comprise configuring the at least one treatment fiber to apply at least two types of treatment to the tissue (stage 352) and generating the signal with respect to the applied treatment type (stage 336). Method 300 may comprise generating the signal with respect to obtained or measured parameters applicable to each of the treatment types (stage 354). For example, the types of treatment may comprise tissue welding and/or tissue cutting.

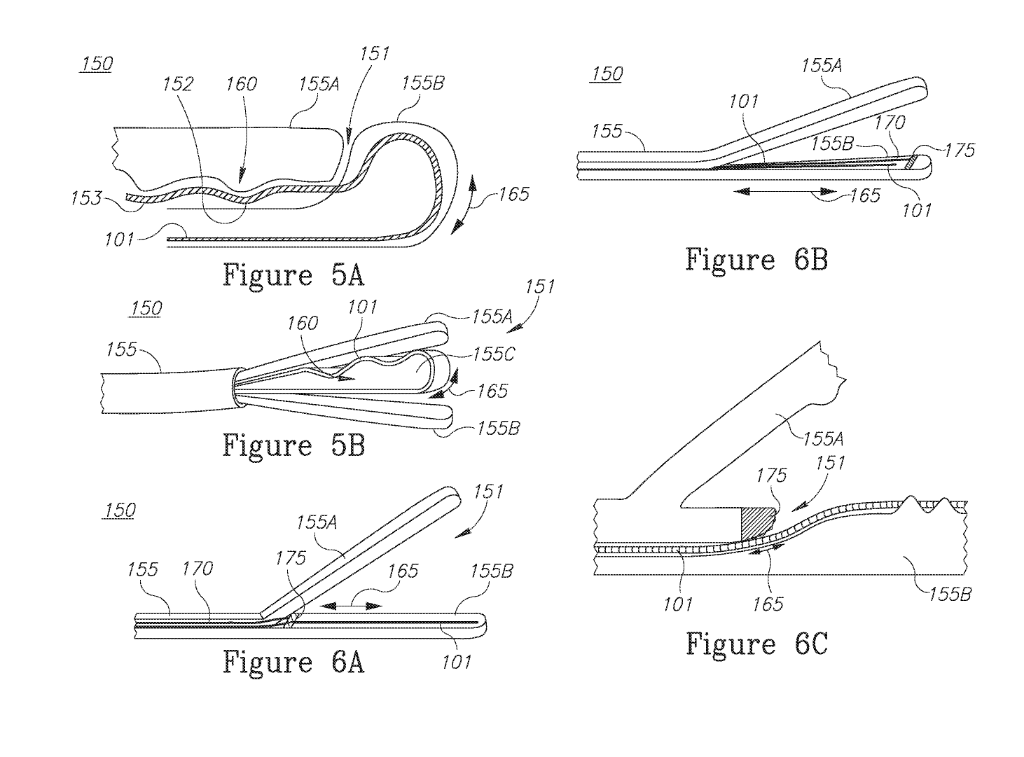

FIGS. 5A, 5B, 6A-6C and 7A-7D are high level schematic illustrations of devices 150 for cleaning and/or regenerating the treatment tip, according to some embodiments of the invention. Advantageously, the illustrated devices prevent the degradation in performance of heat energy based cutting and/or sealing tools due to tissue debris and char that stick to the tool surfaces and require frequent intraoperative cleaning. Illustrated exemplary devices 150 regenerate or replace the active parts of the tool. For example, the active parts may be removed from the active position in the tool and replaced by fresh active parts, or a cleaning mechanism may regenerate the active tool parts in situ.

In certain embodiments, the pressure applied via device 150 may be used to determine the treatment type, e.g., relatively low pressure may be used to apply a sealing treatment while a higher pressure may be used to apply a cutting treatment. The connection between pressure application and treatment type may be direct and mechanical, may be controlled by the degree of fiber bending (e.g., by configuring the bending thresholds of the fiber), may be controlled using measurements from a sensing element (e.g., via sensing signal 92) or the fiber itself, and/or be controlled in an open loop via signals 110 emitted by marking element(s) 120. The applied pressure may be automatically adjusted by control unit 115 in association with device 150 (e.g., with surgical forceps 155).

Device 150 comprises a treatment tool 101 having a treatment tip 152 arranged to apply a treatment to a tissue, wherein a treatment efficiency of treatment tip 152 degrades during the treatment, e.g., due to accumulation of debris and/or tissue fluids, or due to chemical, biological or physical interactions with the tissue. For example, treatment tool 101 may be an optical fiber 101. In certain embodiments, fiber 101 may be attached to a surgical forceps 155 comprising at least two jaws 155A, 155B. In certain embodiments, at least one of jaws 155A, 155B may be indented (see indentation 160 in FIG. 5A). In device 150, treatment tip 152 may be either mobile or stationary, and tip regeneration may be provided wither by stationary parts (e.g., jaw 155B receiving fiber 101) or by mobile parts (e.g., wiper mechanism 170).

Device 150 further comprises a regeneration module 151 arranged to recover the treatment efficiency of treatment tip 152. Regeneration module 151 may be arranged to position a fresh fiber portion 153 as the treatment tip in place of degraded treatment tip 152 to recover the treatment efficiency. Fresh fiber portion 153 comprises a fiber portion which was not used as a treatment tip prior to its positioning or a fiber portion that has been regenerated earlier (see below).

In certain embodiments, regeneration module 151 may be arranged to remove (arrow 165) degraded treatment tip 102 into one of two jaws 155A, 155B of surgical forceps 155 and wherein the respective jaw (e.g., 155B in FIG. 5A) is arranged to receive degraded treatment tip 152. Respective jaw 155B may be arranged to accommodate tip 152 and in certain embodiments regenerate tip 152 to enable repeated use thereof.

In certain embodiments, regeneration module 151 may comprise a central member 155C between two jaws 155A, 155B of surgical forceps 155 which is arranged to replace (arrow 165) degraded treatment tip 152 by fresh fiber portion 153 (FIG. 5B). Central member 155C may be arranged to receive degraded treatment tip 152. Central member 155C may be arranged to accommodate tip 152 and in certain embodiments regenerate tip 152 to enable repeated use thereof. In certain embodiments, central member 155C may be indented (see indentation 160 in FIG. 5B).

In certain embodiments, regeneration module 151 may be arranged to continuously or step-wise rotate fiber 101 in one direction (e.g., one of the directions denoted by arrow 165) to refresh treatment tip 152, e.g., continuously, periodically and/or upon actuation. In certain embodiments, central member 155C may be used to apply different treatments on either side thereof. For example, tissue 80 may be grabbed on one side of member 155C to be welded and to be held and cut on another side of member 155C (see also below, FIGS. 9A-9C).

In certain embodiments, regeneration module 151 may comprise a wiper mechanism 170 arranged to clean and thereby regenerate treatment tip 152 by applying mechanical cleaning, liquid cleaning, gas cleaning and/or chemical cleaning (FIGS. 6A-C, 7A-D). For example, FIG. 6A-6C schematically illustrate a linear wiping arrangement 170 as regeneration module 151. Wiper mechanism 170 comprises a movable pad 175 arranged to regenerate treatment tip 152, e.g., by wiping it mechanically, applying cleansing materials (liquid, gas, with or without chemical activity) thereupon and so forth. Pad 175 may be configured to provide efficient cleaning of treatment tool 101 such as fiber 101. Pad 175 may be made of soft material that preserves the integrity of fiber 101. Pad 175 may be moved at any pattern (e.g. back and forth 165, continuously, rotationally etc.), and be controlled manually, automatically or semi-automatically. Wiper mechanism 170 may be controlled from an operation handle 210 (see FIGS. 9D and 12) of tool 101. Wiper mechanism 170 may be associated with any of jaws 155A, 155B, be combined with jaw 155B arranged to accommodate fiber 101 (FIG. 5A), associated with a central member (FIG. 5B) or with any other configuration of surgical forceps 155.

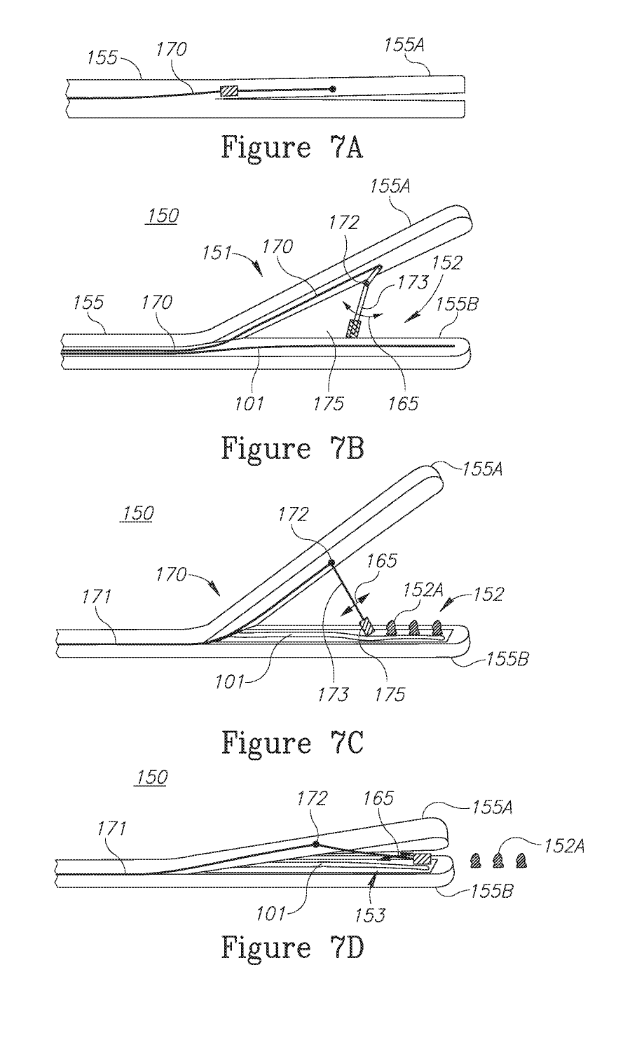

In certain embodiments (FIGS. 7A-D), wiper mechanism 170 may be attached to one of two jaws (e.g., 155A) of surgical forceps 155 and may be arranged to clean fiber 101 attached to the other jaw (e.g., 155B). In the illustrated non-limiting example, pad 175 is mounted on an arm 173 which is movably hinged on a pivot 172. The motion of arm 173 with pad 175 is controlled by wiper mechanism 170, e.g., manually from operation handle 210, automatically, semi-automatically, etc. The area of tool 101 such as fiber 101 may be defined by the angle swept by arm 172 (e.g., wide area treatments such as sealing may require wiping a larger area than narrow area treatments such as cutting, the extent of treated tissue may determine the area that is cleaned, etc.). When regeneration module 151 is not active, it may be withheld in association with the jaw it is mounted upon (e.g., 155A) which may be a jaw without treatment tool 101 such as fiber 101.

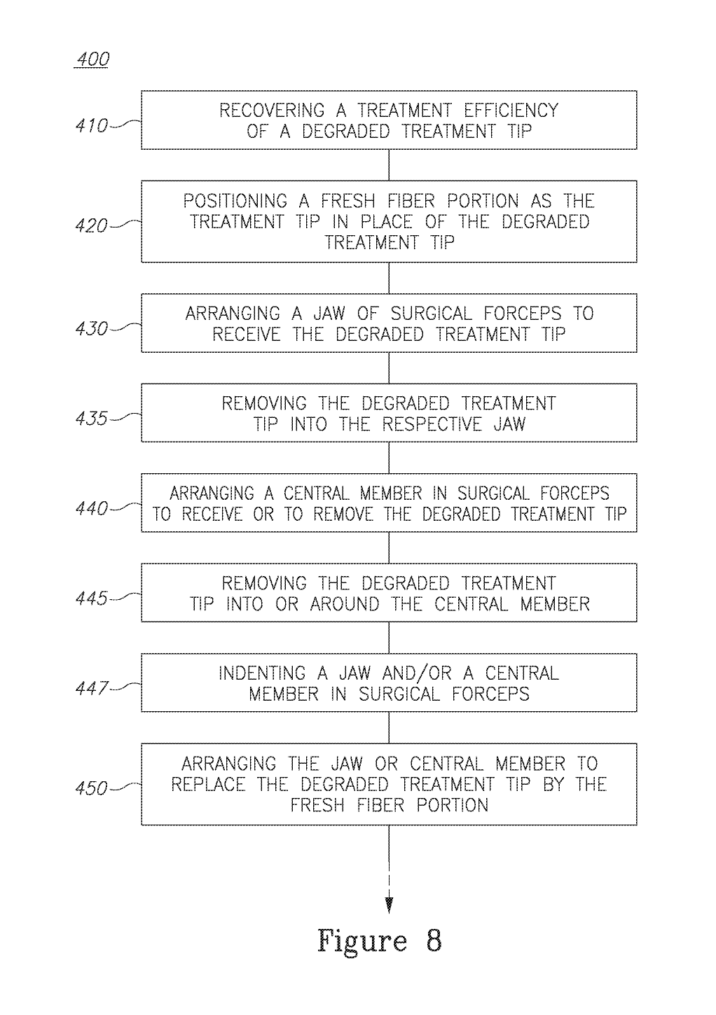

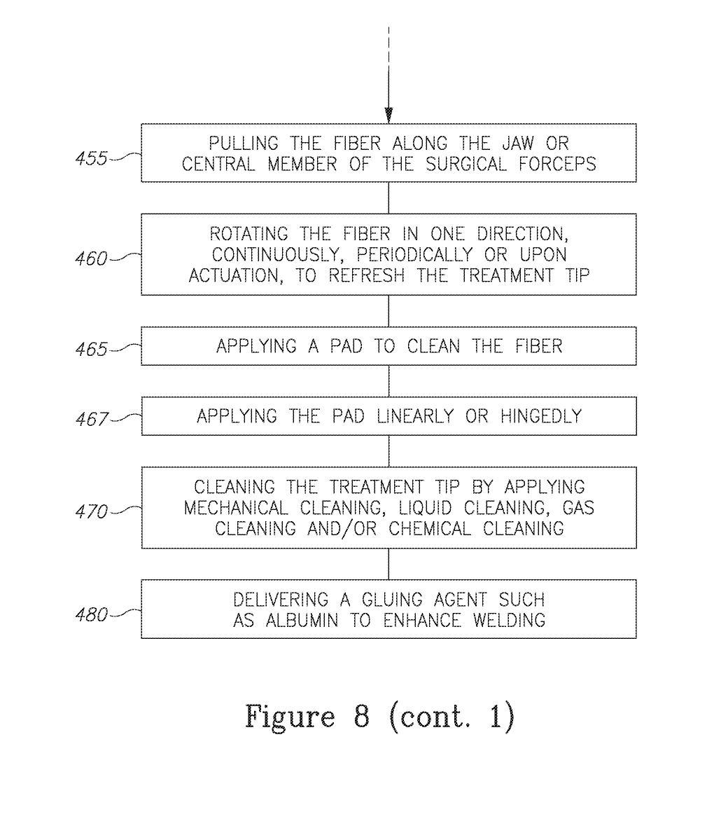

FIG. 8 is a high level schematic flowchart illustrating a method 400, according to some embodiments of the invention. Method 400 comprises recovering a treatment efficiency of a treatment tip (stage 410) arranged to apply a treatment to a tissue, wherein the treatment efficiency of the treatment tip degrades during the treatment. The treatment tool may be an optical fiber and recovering 410 may be carried out by positioning a fresh fiber portion as the treatment tip in place of the degraded treatment tip (stage 420). The fresh fiber portion may comprise a fiber portion which was not used as a treatment tip prior to its positioning or recovered treatment tip. In certain embodiments, method 400 may comprise cleaning the treatment tip by applying mechanical cleaning, liquid cleaning, gas cleaning and/or chemical cleaning (stage 470). Method 400 may further comprise delivering a gluing agent such as albumin to enhance and promote tissue and/or vessel welding (stage 480).

In certain embodiments, the treatment tool may be an optical fiber attached to a surgical forceps and method 400 may further comprise arranging a jaw of the surgical forceps to receive the degraded treatment tip (stage 430) and removing the degraded treatment tip into the respective jaw (stage 435).

In certain embodiments, the treatment tool may be an optical fiber attached to a surgical forceps comprising a central member between jaws thereof and method 400 may further comprise arranging the central member to replace the degraded treatment tip by the fresh fiber portion (stage 440) and removing the degraded treatment tip into the central member (stage 445).

In certain embodiments, method 400 may comprise indenting a jaw and/or a central member in surgical forceps (stage 447). Method 400 may further comprise arranging the jaw or central member to replace the degraded treatment tip by the fresh fiber portion (stage 450). Method 400 may comprise pulling the fiber along the jaw or central member of the surgical forceps 455.

In certain embodiments, method 400 may comprise rotating the fiber in one direction, continuously, periodically or upon actuation, to refresh the treatment tip 460. Method 400 may comprise applying a pad to clean the tool or the fiber (stage 465) and applying the pad linearly (along the tool) or hingedly with respect to a support or a forceps jaw (stage 467).

FIGS. 9A-9E, 10A, 10B, 11A and 11B are high level schematic illustrations of devices 150 for controlling fiber bending, according to some embodiments of the invention.

Device 150 comprises at least one fiber 101 that may be attached to surgical forceps 155 having at least two jaws 155A, 155B. At least one optical fiber 101 is arranged to have at least one specified region that is arranged to emit electromagnetic radiation upon bending optical fiber 155 at the at least one specified region beyond a specified bending threshold. Surgical forceps 155 are arranged to bend, upon actuation, at least one optical fiber 101 at the at least one specified region.

In certain embodiments (FIGS. 9A-9E), surgical forceps 155 may further comprise an element 180 comprising at least a first segment 185A having a first radius of curvature and a second segment 185B having a second radius of curvature (in a non-limiting example, the first radius of curvature is larger than the second radius of curvature). In certain embodiments, element 180 may be egg-like in cross section (FIGS. 9A-C), in certain embodiments, element 180 may be eccentric in cross section. In certain embodiments first segment 185A may be configured to exhibit a first specified curve and second segment 185B may be configured to exhibit a second specified curve, wherein the first and second curves are selected according to requirements concerning the type of treatment applied by segments 185A, 185B, required illumination intensity, etc. In certain embodiments, element 180 may have any non-circular shape in cross section, e.g. a trapezoid (FIGS. 9D-9E). In certain embodiments, element 180 may be held and/or rotated via a supporting arm 154 and may or may not be associated with fiber 101.

Segments 185A, 185B are arranged, upon actuation, to bend at least one fiber 101 to respective bending thresholds. Thus, element 180 may be set to define the radius of curvature of fiber 101 and thus emission from fiber 101. In certain embodiments, the respective bending thresholds may correspond to the first and second radiuses of curvature.

In certain embodiments, element 180 may be arranged to affect either or both treatment fiber(s) 101 and marking fiber(s) 120. For example, bending controlled emission may be carried out with respect to treatment fiber 101 and/or marking fiber 120. In certain embodiments, one or more of fibers 101 may be arranged to emit treatment radiation 90, marking signal 110 and/or sensing signal 92. Forceps 155 may be arranged to direct signal 110 onto tissue 80 to be visible to the surgeon. In certain embodiments, element 180 may be rotatable (e.g. by an actuator 187 on handle 210, see FIG. 9D) to allow control of the applied bending radius and hence of the emission from fiber(s) 101.

In certain embodiments, segments 185A, 185B may be arranged, upon actuation, to press tissue 80 to different extent, to control the tissue area onto which treatment is applied. Element 180 may be applied to control fiber curvature and/or tissue pressing by itself or in combination with jaws 155A, 155B (see FIGS. 9D and 9E) and/or supports 145A, 145B (see FIGS. 3A and 3B above).

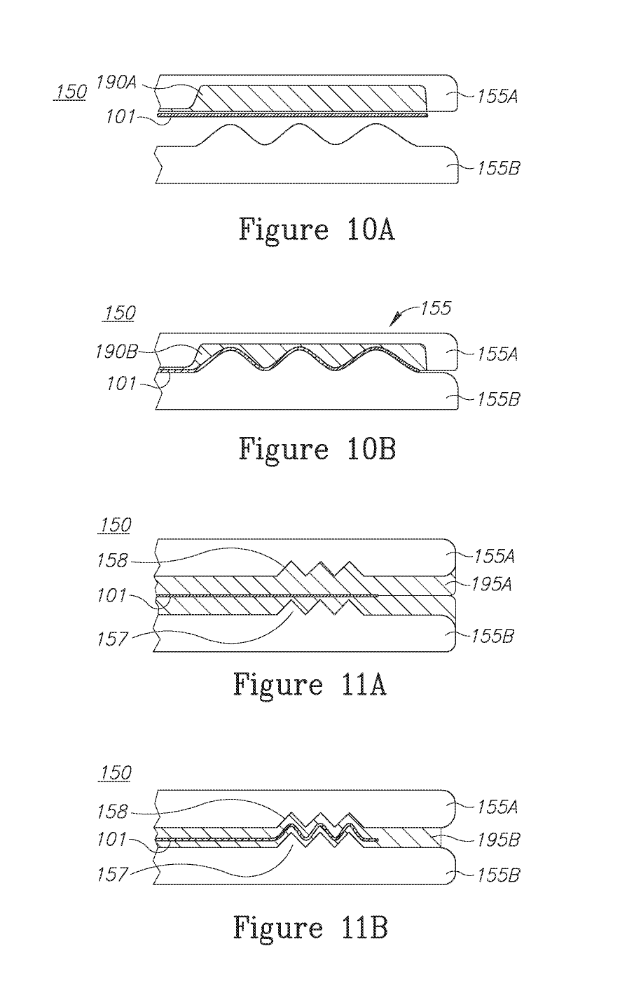

In certain embodiments (FIGS. 10A, 10B, 11A and 11B), one or more forceps jaws 155A, 155B may comprise at least one protrusion 157 and optionally at least one corresponding recess 158 configured to bend at least one optical fiber 101 to the specified bending threshold. The design of jaws 155A, 155B may be configured to bend fiber(s) 101 at specified bending radiuses configured to apply respective treatments and/or emit respective signals (e.g., modulate emission type and intensity).

In certain embodiments, one or both jaws 155A, 155B may fiber-supporting material configured to support fiber bending and or the treated tissue. For example, FIGS. 10A and 10B schematically illustrate protrusions 157 on jaw 155B and fiber-supporting material 190A on jaw 155A. Upon actuation, jaw 155B may be pressed against jaw 155A, resulting in fiber bending which is backed by yielding fiber-supporting material 190B, which takes a specified form. The fiber supporting material may be arranged to support treated tissue and allow it to comply to jaw 155B with fiber 101. In another example, FIGS. 11A and 11B schematically illustrate fiber 101 embedded within or surrounded by fiber-supporting material. In the illustrated example, fiber 101 does not contact any of jaws 155A, 155B but is deformed together with fiber-supporting material 195A into bended configuration within fiber-supporting material 195B. Tissue 80 may be inserted between the jaws to contact fiber-supporting material 195A with fiber 101 embedded within or attached to fiber-supporting material on either or both jaws 155A, 155B.

FIGS. 12A and 12B are high level schematic illustrations of safety assemblies 183, according to some embodiments of the invention. In FIGS. 12A and 12B safety assembly 183 is exemplified in a non-limiting manner for surgical forceps 155. Safety assembly 183 may be implemented according to similar principles in any of devices 100 and 150. For example, safety assembly 183 may comprise supports 183A, 183B arranged to protect the practitioner from laser radiation and to ensure the treatment efficiency. Supports 183A, 183B may comprise respective radiation absorbing material 182A, 182B, for example selected to absorb in the wavelength range of the laser used in treatment tool 101. Supports 183A, 183B may comprise respective elements 181A, 181B configured to detect failure of radiation absorbing material 182A, 182B and possibly stop energy delivery upon such detection to assure operation safety of tool 101. Supports 183A, 183B may be configured to have geometrical forms that control the degree of bending of fiber 101 and/or that limit or define the treatment application area. The geometrical forms of supports 183A, 183B may be complementary, for example, as illustrated in FIGS. 12A and B, support 183A may comprise a protrusion fitting into a cavity in support 183B which hold optical fiber 101 and accommodate a treated portion of tissue 80. The cavity may be shielded from the surrounding environment by lateral parts of supports 183A, 183B.

FIG. 13A is a high level schematic block diagram of a cordless surgical laser unit 200, according to some embodiments of the invention. In FIG. 13A, optical connections are indicated by the reference numeral 201 while electric connections are indicated by the reference numeral 202. Cordless surgical laser unit 200 may comprise a disposable unit 230 comprising surgical mechanical head 250 (e.g., devices 100 or 150, comprising e.g., forceps 155, glove 140, the handheld device illustrated in FIGS. 3B, 3C or another device), optical fiber 101 connected to an optical/electrical connector 232, as well as suction 234 and light source 120 as marking element 120 or as a light source for illuminating the treatment location and its surroundings. Optical fiber 101 may be incorporated in surgical mechanical head 250 as illustrated in the figures presented above. Disposable unit 230 is connected optically and electrically to a reusable handle 210, comprising a rechargeable and/or disposable power source 212 with an optional cooling unit 214, as well as control unit 115, laser source 224 and optics arranged to deliver treatment radiation to fiber 101.

Advantageously, the use of fibers 101 as treatment tools, and even more so treatment by emission only upon fiber bending, allows using less power and consequentially implementing treatment fiber 101 as a handheld unit having reusable handle 210 and disposable fiber unit 230.

In certain embodiments, power source 212 may be configured to generate between 10 W and 60 W and cooling unit 214 may be configured to cool power source 212. Power source 212 as a battery may comprise least between 10-50 Watt hours. At typical laser diode wavelengths (for example 980 nm, 1064 nm, 1470 nm, etc.), about 10 W output optical power are required. Assuming typical laser efficiency of 85% and typical electric efficiency of 35%, a power supply of 60 W would generate heat at 24 W. Such heat generation requires cooling to keep power source 212 and laser source 224 at working temperatures. Cooling by unit 214 may be carried out passively or actively, in the latter case by a fan, by compressed air or thermoelectrically. For example, assuming 120 W for cooling, a total power demand of 156 W may be satisfied by a 25 Watt hour battery, enabling the handheld configuration.

FIG. 13B is a high level schematic block diagram of treatment devices 100 and/or 150, according to some embodiments of the invention. The various configurations and combinations of the features of devices 100 and/or 150 which are schematically illustrated in FIG. 13B are part of the present disclosure. Devices 100 and/or 150 may comprise any or all of treatment tool 101, control unit 115 and marking element(s) 120 as described above. Marking element(s) 120 may be applied to any standard surgical device. Devices 100 and/or 150 may further comprise associated manipulation structures such as glove 140, surgical forceps 155, placing template 145 with corresponding supports, jacket 107, safety assembly 183, regeneration module 151, element 180 having varying radiuses of curvature etc. each for itself or any feasible combination of these features. Marking element(s) 120 is arranged to apply signal 110 onto tissue 80, treatment tool 101 is arranged to apply energy (e.g., electromagnetic, mechanical, electrical, thermal) to tissue 80, and the associated manipulation structures may apply pressure to tissue 80, regenerate treatment tool 101, provide spatial positioning assistance, enhance the handling simplicity or operation of treatment tool 101 and tissue 80 etc. as described above. Devices 100 and/or 150 may further comprise sensing unit(s) 93 and optionally external unit(s) 94 that provide parameters (e.g., patient, device, treatment or tissue parameters) to control unit 115. The parameters may be obtained passively (e.g., patient or device data) or actively (e.g., realtime measurements of tissue and treatment parameters using sensing signal 92). Parameters may also be received from treatment tool 101. Control unit 115 controls marking element(s) 120 and may also control treatment tool 101 and/or the associated manipulation structures with respect to their operation (e.g., electromagnetic, mechanical, electrical, thermal).

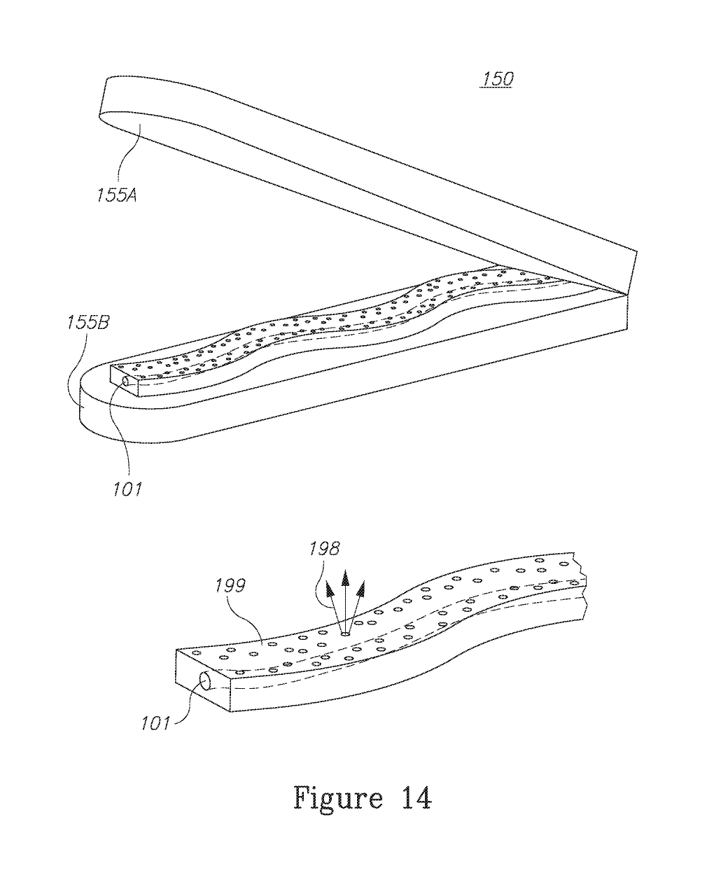

FIG. 14 is a high level schematic illustration of welding-promoting device 150, according to some embodiments of the invention. Device 150 such as surgical forceps having two jaws 155A, 155B (or alternatively, glove or hand supports), comprises a perforated member 199 associated with (e.g., enclosing) treatment fiber 101. Perforated member 199 may be arranged to deliver a welding promoting fluid 198 such as an adhesive or a welding promoting protein (e.g., an albumin solution) that promotes tissue welding, vessel welding or generally enhances the applied treatment.

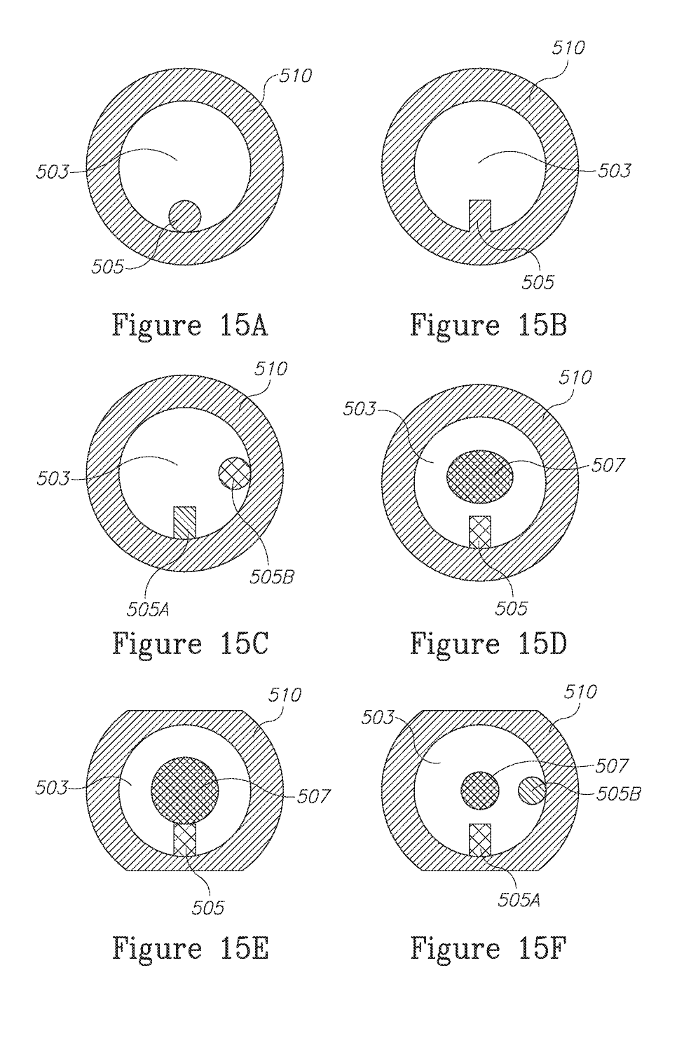

FIGS. 15A-15F are high level schematic illustrations of MCVD (modified chemical vapor deposition) fiber production methods, according to some embodiments of the invention. FIGS. 15A-15F present deposition walls 510 as supports for performing the MCVD process (e.g., as tube walls 510). It is noted that walls 510 may be removed after completion of the fiber production, or may be used, at least in part, as components of the optical fiber (e.g., as coating cladding or even parts of the core). One or more emitting regions 505 may be attached (e.g., fused or deposited) or positioned on the inside of wall 510 prior to production of the rest of the fiber, or may be designed as part of wall 510. In certain embodiments, elements 505 may be fillers used for later production (e.g., by attachment, deposition or placing) of emitting regions after removing fillers 505. Emitting region(s) 505 may be placed as respective rod(s) made of different shapes and/or materials inside deposition tube wall 510, prior to the vapor deposition. Emitting regions 505 of different forms (FIGS. 15A, 15B) and possibly multiple emitting regions (e.g., 505A, 505B) with similar or different characteristics may be placed or designed as part of wall 510 (FIGS. 15C, 15F). Inner void 503 of wall 510 may be used to produce fiber 101 by MCVD, wherein the fiber's core and possibly at least part of the fiber's cladding may be produced in void 503. In certain embodiments, a core 507 may be central or eccentric, may be round or elliptic (FIG. 15D) and may or may not contact emitting region(s) 505 (FIGS. 15E, 15F respectively). Wall 510, especially when being retained as part of the produced fiber (e.g., as coating or cladding), may be not round, e.g., flattened or have edges cut away (FIGS. 15E, 15F) to yield fiber 101 with radially varying width. The illustrated production methods may be applied to any one of the fiber embodiments described above. It is noted that emitting region(s) 505 may be fused with wall 510 or may be set within wall 510 without fusing. It is also noted that the materials of the elements may be the same, similar, or different, and generally be selected according to specified performance requirements. Specifically, material deposited during the MCVD process may have a lower index refraction or a higher index of refraction with respect to wall 510 or may be composed of different materials with different indices of refraction, possible having layers of lower indices and higher indices.

In certain embodiments, after applying one or more layers by MCVD, the resulting coated wall may be collapsed and processed into a preform (such as a rod), e.g., by at least partially removing wall 510, possibly in a radially asymmetric manner to produce facets on the preform than result in a radially asymmetric fiber (FIGS. 15E, 15F), and then the preform may be drawn to form a fiber, which may then be coated.

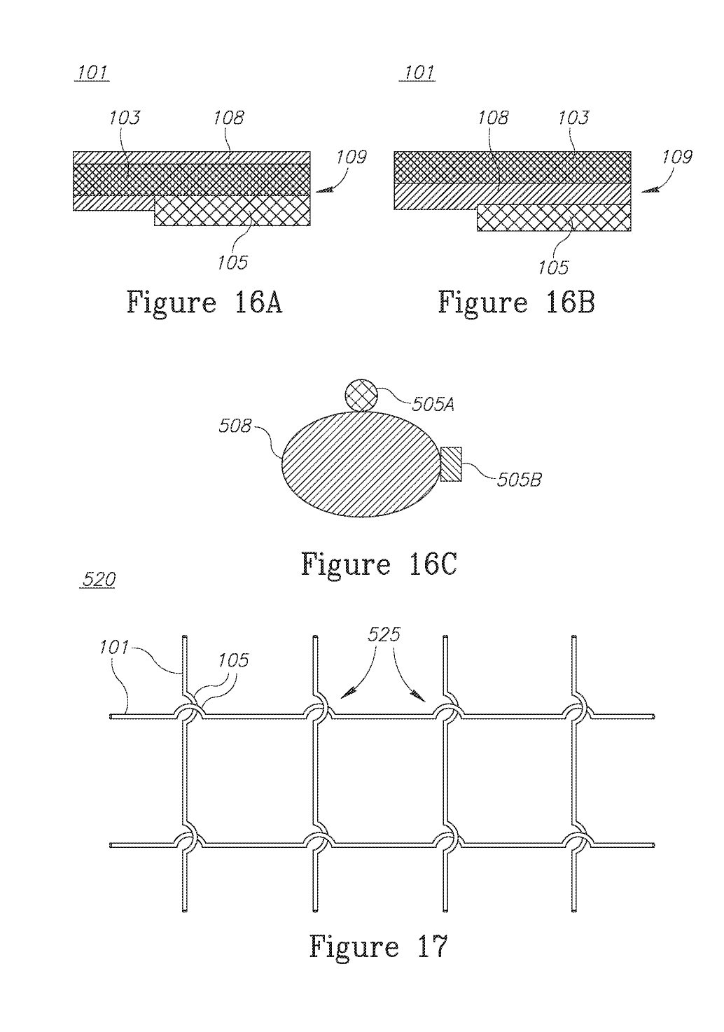

FIGS. 16A-16B are high level schematic illustrations of optical fibers 101 produced from coreless coated fibers, according to some embodiments of the invention. FIGS. 16A, 16B illustrate longitudinal cross sections of a part of fiber 101 (and/or a preform thereto). The coreless coated fibers may comprise a coating 108 and a central member 103 which may be a void or a core. In certain embodiments, one or more emitting regions 105 may replace a region of coating 108 (FIG. 16A) or may be attached upon a region of coating 108 (FIG. 16B). In either case, emitting region(s) 105 may be attached at a tip 109 of fiber 101 or may be configured along fiber 101, possibly along a substantial length of fiber 101 or recurring at intervals along fiber 101. For example, the coreless fiber may be coated with a lower index coating 108. Coating 108 may be removed at distal tip 109 and a thin slab or window of glass fiber may be welded as emitting region 105 in place of the removed coating, to result in a terminal emitting region 105 if fiber 101 after drawing.

FIG. 16C is a high level schematic illustration of a preform for optical fibers 101, the preform produced by outside vapor deposition (OVD) fabrication method, according to some embodiments of the invention. Fibers 101 may be produced from preforms to which one or more emitting region(s) 505A, 505B, possibly made of different materials and possibly have different forms, may be attached, fused or deposited, e.g., by OVD, to a central member 508 (which may comprise the preform for the fiber core and possible for the fiber cladding).

FIG. 17 is a high level schematic illustration of a sheet 520 of woven optical fibers 101, according to some embodiments of the invention. Optical fibers 101 may be of the above embodiments and specifically may each comprise one or more (e.g., a plurality of) emitting region(s) 105 configured to emit transferred electromagnetic radiation from a core through a cladding of optical fiber 101 upon bending of optical fiber 101 at emitting region(s) 105 beyond a specified bending threshold. For example, emitting region(s) 105 may be bended at fiber crossings 525 to emit radiation which is focused on a point external to sheet 520 of woven fibers 101. In certain embodiments, emitting region(s) 105 may be arranged at fiber crossings 525. Sheets 520 may be used to apply a surface treatment composed of an array of treatment loci to a tissue.

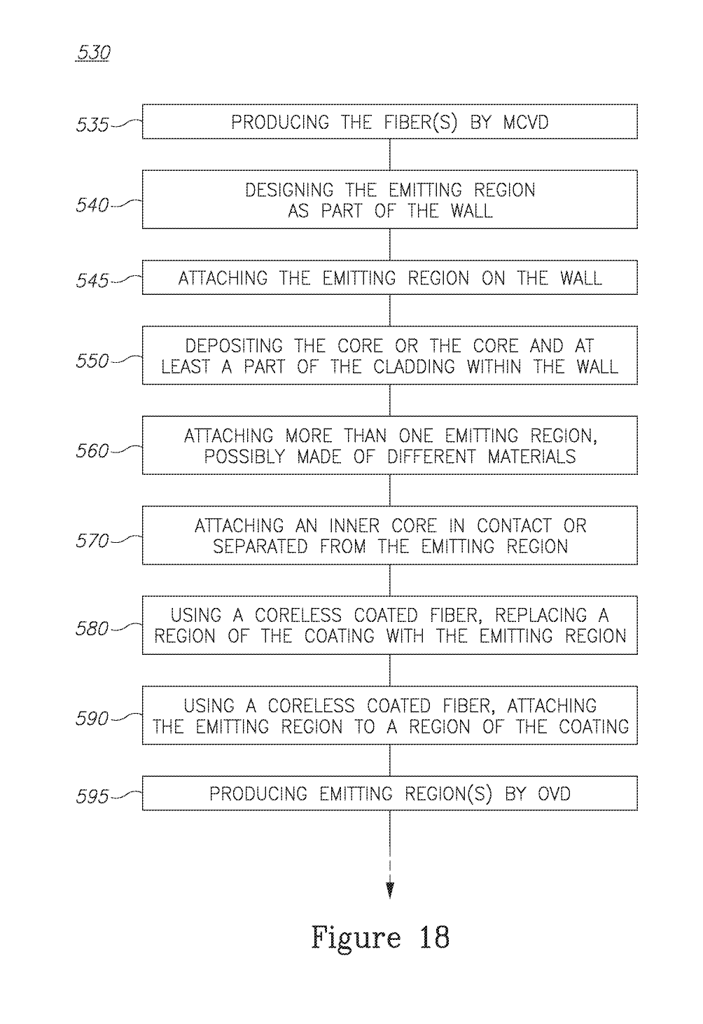

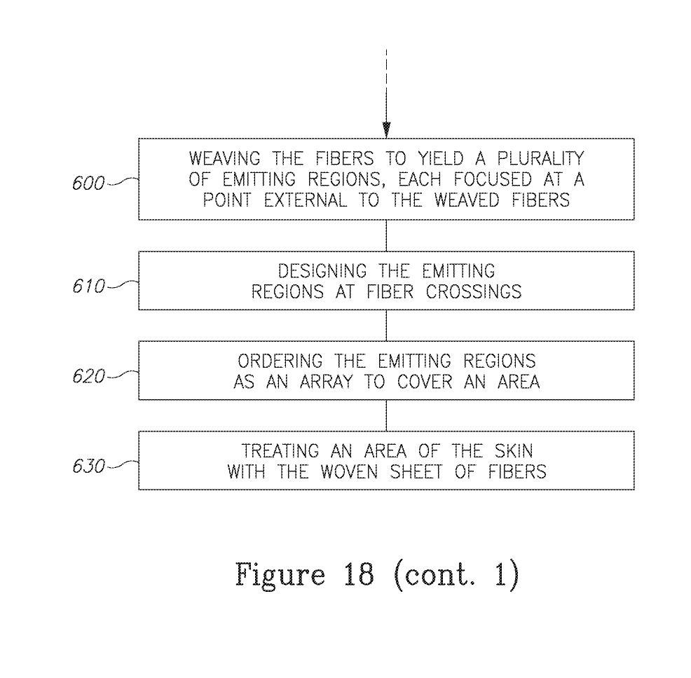

FIG. 18 is a high level schematic flowchart illustrating a method 530, according to some embodiments of the invention. Method 530 may be configured in certain embodiments to produce fibers 101 and fiber fabric 520. Method 530 may comprise producing at least one emitting region 105 of optical fiber 101, wherein emitting region(s) 105 is configured to emit transferred electromagnetic radiation from the fiber's core through the fiber's cladding upon bending the optical fiber at the emitting region(s) beyond a specified bending threshold. Certain embodiments comprise optical fibers 101 produced by any embodiments of method 530.

Method 530 may comprise producing the fiber(s) by MCVD (stage 535), e.g., comprising designing the emitting region as part of the wall (stage 540), attaching (e.g., fusing, depositing or positioning) the emitting region on the wall (stage 545), depositing the core or the core and at least a part of the cladding within the wall (stage 550), attaching more than one emitting region, possibly made of different materials (stage 560) and/or attaching (e.g., placing) an inner core in contact or separated from the emitting region (stage 570).

In certain embodiments, method 530 may comprise using a coreless coated fiber, replacing a region of the coating with the emitting region (stage 580) and/or using a coreless coated fiber, attaching the emitting region to a region of the coating (stage 590). Certain embodiments comprise producing emitting region(s) by OVD (outside vapor deposition) (stage 595), e.g., by attaching the emitting region(s) externally to a respective preform.

Method 530 may further comprise weaving the fibers to yield a plurality of emitting regions, each focused on a point external to the weaved fibers (stage 600), designing the emitting regions at fiber crossings (stage 610), ordering the emitting regions as an array to cover an area (stage 620) and optionally treating an area of the skin with the woven sheet of fibers (stage 630).

To summarize, and without limiting the scope of the invention, certain embodiments comprise the following aspects: (i) Visual marking on treated tissue which indicates any of various parameters relating to the patient, the treatment, the treatment tool, treatment efficiency etc. (ii) Laser eye safety elements which are integrated into the treatment tool in various configurations. (iii) Finger held treatment devices and glove-implemented treatment devices that allow manipulation of the treatment tool such as an optical fiber with the fingers, with associated safety configurations. (iv) Tip cleaning mechanisms arranged to clean, replace or refresh a treating tip of a treatment toot, such as an active region of an optical fiber. (v) Tool tips which apply soldering material (e.g., biological materials) to enhance tissue welding. (vi) Treatment tips which are shaped and designed to control the pressure applied on the treated tissue and/or optical fibers in the treatment tip (as well as fiber bending degrees) to yield specified treatment effects. (vii) Device configurations as battery operated-handheld laser surgical tools. (viii) Fiber production possibilities such as MCVD or using coreless fibers, as well as fiber weaving applications. Any combinations of these aspects and their exemplary embodiments presented above are part of the present disclosure.

In the above description, an embodiment is an example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A device comprising:
a treatment tool arranged to apply a treatment to a target,
a control unit arranged to obtain or measure at least one parameter, and
at least one marking element associated with the treatment tool and arranged to illuminate the target with a signal generated by the control unit and relating to the at least one obtained or measured parameter.

2. The device of claim 1, wherein the control unit is further arranged to measure at least one parameter indicative of an efficiency of the treatment, and the at least one marking element is further arranged to indicate the measured at least one parameter.

3. The device of claim 1, wherein the treatment tool is at least one treatment fiber.

4. The device of claim 1, wherein the treatment tool is at least one treatment fiber and wherein the at least one marking element is at least one marking fiber which is implemented together with the at least one treatment fiber as a single fiber.

5-13. (canceled)

14. The device of claim 1, wherein the signal relates to the at least one obtained or measured parameter by at least one of: a signal color, a signal intensity, a signal periodicity, a direction of the signal.

15. The device of claim 1, wherein the signal relates to the at least one obtained or measured parameter by a temporal change of the signal.

16-17. (canceled)

18. The device of claim 1, wherein the control unit is further arranged to generate the signal with respect to a treatment type applied by the treatment tool.

19. (canceled)

20. The device of claim 1, wherein the at least one obtained or measured parameter comprises at least one of: a target parameter a device parameter and a patient parameter.

21. (canceled)

22. (canceled)

23. The device of claim 1, wherein the at least one obtained or measured parameter comprises at least one of: a target temperature, index of refraction at a target interface, a target impedance and Doppler shift measurements of the target or of target fluids.

24. The device of claim 1, wherein the at least one obtained or measured parameter comprises at least one of: operational parameter of the device, device failure and device readiness.

25. (canceled)

26. The device of claim 1, wherein the at least one marking element comprises at least one light emitting diode (LED), at least one fiber and/or at least one projection device.

27. (canceled)

28. The device of claim 1, wherein the treatment tool comprises at least one of: a passive treatment tool, an electric treatment tool, a laser treatment tool and an ultrasonic treatment tool.

29. A method comprising:
associating at least one marking element with a treatment tool that is arranged to apply a treatment to a target, and
configuring the at least one marking element to illuminate the target with a signal.

30. A method of indicating an efficiency of a treatment to a target by a treatment tool, the method comprising:
measuring at least one parameter,
generating a signal relating to the at least one obtained or measured parameter, and
illuminating the target with the signal by at least one marking element.

31-37. (canceled)

38. The method of claim 30, further comprising relating the signal to the at least one obtained or measured parameter by at least one of: a signal color, a signal intensity, a signal periodicity, a direction of the signal.

39. The method of claim 30, further comprising relating the signal to the at least one obtained or measured parameter by a temporal change of the signal.

40. The method of claim 30, further comprising configuring the signal to dynamically illuminate the target.

41. The method of claim 30, further comprising configuring the illumination of the target to relate to the at least one obtained or measured parameter by at least one of: an extent of the illumination, an intensity of the illumination, a color of the illumination, a region of the illumination.

42-100. (canceled)
